(19) United States
(12) Patent Application Publication
(10) Pub. No.: US 2003/0108920 A1
(43) Pub. Date: Jun. 12, 2003

(54) Tumor suppressor-like proteins that bind IGFBP2

(76) Inventors: Wei Zhang, Houston, TX (US); Wei Sonya Song, Houston, TX (US); Greg Fuller, Houston, TX (US)

Correspondence Address: Priya D. Subramony, Fulbright & Jaworski L.L.P., Suite 2400, 600 Congress Avenue, Austin, TX, 78701, US

(21) Appl. No.: 10/237,566

(22) Filed: Sep. 9, 2002

Related U.S. Application Data
(60) Provisional application No. 60/318,200, filed on Sep. 7, 2001.

(51) Int. Cl. A61K 48/00; A61K 38/17; A01K 67/027; C12Q 1/68; G01N 33/574; C12P 21/02; C12N 5/06; C07K 14/47

(52) U.S. Cl. 435/6; 435/7.23; 435/69.1; 435/320.1; 435/325; 514/44; 514/12; 530/350; 536/23.2; 800/18

(57) ABSTRACT

The present invention provides the identification of a tumor suppressor protein that binds to IGFBP2. This 388 amino acid protein, designated 86, contains an ICE-p20 domain, and is expressed in a number of normal tissues, include spleen, placenta, stomach, brain (including cerebellum) and liver. It also is expressed, ocassionally with a splice variant lacking a single exon, in various cancer cells including gliomas. Thus, methods for diagnosing and treating cancers having defects in the 86 gene are disclosed, as are methods for screening compounds for their ability to modulate 86 expression and function.

The present application claims priority to co-pending U.S. patent application Ser. No. 60/318,200, filed Sep. 7, 2001. The entire text of the above referenced disclosure is specifically incorporated by reference herein without disclaimer.

### BACKGROUND OF THE INVENTION

1. Field of the Invention

The present invention relates to the field of oncology, genetics, and molecular biology. More specifically, the invention relates to the identification of a tumor suppressor gene on chromosome 1p36, a region that is frequently deleted in a common form of brain tumor oligodendrogliomas, that binds Insulin-Like Growth Factor Binding Protein 2 (IGFBP2).

2. Related Art

Oncogenesis was described by Foulds (1958) as a multistep biological process, which is presently known to occur by the accumulation of genetic damage. On a molecular level, the multistep process of tumorigenesis involves the disruption of both positive and negative regulatory effectors (Weinberg, 1989). The molecular basis for human colon carcinomas has been postulated, by Vogelstein and coworkers (1990), to involve a number of oncogenes, tumor suppressor genes and repair genes. Similarly, defects leading to the development of retinoblastoma have been linked to another tumor suppressor gene (Lee et al., 1987). Still other oncogenes and tumor suppressors have been identified in a variety of other malignancies. Unfortunately, there remains a large number of poorly or untreatable cancers, and the effects are catastrophic with over half a million deaths per year in the United States alone.

Glioma-glioblastoma multiforme (GBM) is the most malignant of the neuroepithelial neoplasms, characterized by cellular pleomorphism, numerous mitotic figures, and often multinucleated giant cell. The tumor is usually solid, although cysts may be present. Rarely the tumor consists of a solitary cyst and mural nodule. GBM constitutes approximately 7% of childhood intracranial neoplasms. Childhood glioblastomas of the cerebral hemispheres are also located most often in the frontal lobe; with the second most frequent site being the parietal lobe. Primary glioblastoma of the spinal cord in childhood is rare.

Glioblastoma multiforme in children appears to have two characteristic courses, each of which is related to the location of the tumor. Glioblastomas of the brainstem, a more primitive part of the central nervous system, occur at a younger age and have a shorter mean survival relative to those of the cerebral hemispheres. Glioblastoma multiforme of the cerebral hemisphere, a more highly developed part of the central nervous system, is characterized by onset in older children (13 years) and by a longer mean survival.

Survival rates in patients with glioblastoma multiforme is uniformly poor. In studies of children treated with surgery and intracranial radiation, only one third of the children are alive one year after diagnosis. Survival of children with glioblastoma multiforme of either of the cerebral hemispheres or the brainstem has significantly increased since the advent of dexamethasone therapy. Presently therapy consists of surgery plus combination chemotherapy (8 in 1).

Insulin-Like Growth Factors (IGFs) are widely reported to have a profound effect on the growth and differentiation of normal and malignant cells (Hwa et al., 1999). In addition, six IGF-binding proteins (IGFBP1-6) have been found to bind IGFs with high affinity and then modulate IGF activities (Martin and Baxter, 1999). Several studies suggest that IGFBPs are also involved in other, IGF-independent, cellular processes (Oh et al., 1993). IGFBP2 is reported to be over expressed in the most advanced stage of GBM (Fuller et al., 1999). The overexpression of IGFBP2 also has been reported in the advanced stages of ovarian cancer, prostate cancer, colon cancer, and breast cancer (Karasik et al., 1994; Kanety et al., 1996; Ho and Baxter, 1997; Kanety et al., 1993; Mishra et al., 1998; Renehan et al., 2000; Manni et al., 1994; Wex et al., 1998). The fact that IGFBP2 is over expressed in many advanced stage tumors suggests that it may have a role in cancer progression.

Cytogenetic aberrations, as well as high frequency loss of heterozygosity (LOH), have been observed within the short arm of human chromosome 1 (Bomme et al., 1994; Bieche et al., 1994; Kovacs et al., 1988; Bieche et al., 1998). Deletion of the short arm of chromosome 1 is seen in the majority of oligodendroglial tumors (Smith et al., 2000; Bello et al., 1995; Reifenberger et al., 1994; Nigro et al., 2001; Ino et al., 2000). At least three separate regions of LOH have been consistently documented with chromosome 1p (1p22-1p31, 1p34-1p35 and 1p36) in a variety of histologically diverse human tumors, including breast, colon and neuroblastoma (Bomme et al., 1994; Bieche et al., 1994; Kovacs et al., 1988; Bieche et al., 1998; Lo Cunsolo et al., 1999). LOH in familial breast cancer indicated common regions of loss that included 1p36 (32%) and 1p32 (51%) (Millikan et al., 1999).

A recent report investigated LOH in a variety of solid tumors and found high frequency LOH in stomach, colon and rectum, breast, endometrium, ovary, testis, kidney, thyroid and sarcomas (Ragnarsson et al., 1999). In addition, several studies have shown that deletions in the 1p36 and 1p32 region correlated with poor survival in colon and breast cancers (Borg et al., 1992; Ogunbiyi et al., 1997). Functional studies using microcell fusion also have mapped a tumor suppressor locus in colon cancer to within chromosome 1p36 (Tanaka et al.,1993). Candidate tumor suppressor genes p73 and Rad54 have been mapped to 1p36 and 1p32, respectively. However, expression studies and mutational analyses have failed to confirm their importance in the biology of colon and breast cancers (Han et al., 1999; Ichimiya et al., 1999; Rasio et al., 1997). Thus, these data suggest that an important yet unidentified tumor suppressor gene or genes resides within chromosome 1p32-1p36 and is involved at high frequency in a number of histologically diverse human cancers.

Despite all of this information, the identity of the gene or genes involved with chromosome 1 LOH remains elusive. Without identification of a specific gene, and deduction of the protein for which it codes, it is impossible to begin developing an effective therapy targeting this product. Thus, it is an important goal to identify the tumor suppressor(s) located in this region.

### SUMMARY OF THE INVENTION

Thus, in a first aspect of the invention, there is provided an isolated polypeptide comprising an amino acid sequence of SEQ ID NO: 1, SEQ ID NO: 2 or SEQ ID NO: 3.

In another aspect of the invention, there is provided a polynucleotide encoding a polypeptide having the amino acid sequence of SEQ ID NO: 1, SEQ ID NO: 2 or SEQ ID NO: 3. In particular embodiments, the polynucleotide sequence is represented by SEQ ID NO: 4, SEQ ID NO: 5 and SEQ ID NO: 6, respectively. The polynucleotide may further comprise a promoter operable in eukaryotic cells, for example, a promoter that is heterologous to the coding sequence. Such a promoter could be Hsp68, SV40, CMV, MKC, GAL4_{UAS}, HSV or β-actin. Alternatively, the promoter can be a tissue specific promoter or an inducible promoter.

Also provided is an expression cassette comprising a polynucleotide encoding a polypeptide having the sequence of SEQ ID NO: 1, SEQ ID NO: 2 or SEQ ID NO: 3, wherein the polynucleotide is under the control of a promoter operable in eukaryotic cells. The expression cassette may be contained in a viral vector, such as a retroviral vector, an adenoviral vector, an adeno-associated viral vector, a vaccinia viral vector, or a herpesviral vector. The expression cassette may further comprise a polyadenylation signal. The expression cassette may further comprise a second polynucleotide encoding a second polypeptide, optionally under the control of a second promoter.

In yet another embodiment, there is provided a method for suppressing growth of a cancer cell comprising contacting the cells with an expression cassette comprising a polynucleotide encoding a polypeptide having the sequence of SEQ ID NO: 1, SEQ ID NO: 2 or SEQ ID NO: 3, wherein the polynucleotide is under the control of a promoter operable in eukaryotic cells.

In still yet another embodiment, there is provided a cell comprising an expression cassette comprising a polynucleotide encoding a polypeptide having the sequence of SEQ ID NO: 1, SEQ ID NO: 2 or SEQ ID NO: 3, wherein the polynucleotide is under the control of a promoter operable in eukaryotic cells.

In a further embodiment, there is provided a monoclonal antibody that binds immunologically to a polypeptide having the sequence of SEQ ID NO: 1, SEQ ID NO: 2 or SEQ ID NO: 3, or an immunologic fragment thereof. The antibody may further comprise a detectable label, for example, a fluorescent label, a chemiluminescent label, a radiolabel or an enzyme. Also provided is the corresponding hybridoma cell, and equivalent polyclonal antisera.

In an additional aspect, there is provided a method of diagnosing a cancer comprising the steps of (i) obtaining a tissue sample from a subject; and (ii) assessing the expression of an 86 tumor suppressor in cells of the sample. The cancer may be selected from the group consisting of brain, lung, liver, spleen, kidney, lymph node, small intestine, pancreas, blood cells, colon, stomach, breast, endometrium, prostate, testicle, ovary, skin, head and neck, esophagus, bone marrow and blood cancer, and specifically is a glioblastoma. The cancer may be a carcinoma or a neuroblastoma. The sample may be a tissue or fluid sample. Assessing may comprise assaying for an 86-encoding nucleic acid from the sample, and optionally amplifying the nucleic acid. Alternatively, assessing comprises contacting the sample with an antibody that binds immunologically to an 86 polypeptide, for example, in an ELISA.

The method may involve evaluating the level of 86F expression, for example, comparing the expression of 86F in cancer samples with the expression of 86F in non-cancer samples. The method may involve comparing the expression of 86F, 86Y, and 86S in either cancer or non-cancer samples. The method may also involve evaluating the structure of the 86F gene or transcript. The evaluating may comprise an assay selected from the group consisting of sequencing, wild-type oligonucleotide hybridization, mutant oligonucleotide hybridization, SSCP, PCR and RNase protection. In particular, the evaluating is by wild-type or mutant oligonucleotide hybridization and the oligonucleotide is configured in an array on a chip or wafer.

In a further embodiment, there is provided a method for altering the phenotype of a tumor cell comprising the step of administering to a cell a tumor suppressor designated 86F under conditions permitting the uptake of the tumor suppressor by the tumor cell. The tumor cell may be derived from a tissue selected from the group consisting of brain, lung, liver, spleen, kidney, lymph node, small intestine, blood cells, pancreas, colon, stomach, breast, endometrium, prostate, testicle, ovary, skin, head and neck, esophagus, bone marrow and blood tissue. The phenotype may be selected from the group consisting of apoptosis, angiogenesis, proliferation, migration, contact inhibition, soft agar growth and cell cycling. The tumor suppressor may be encapsulated in a liposome.

In still another aspect of the invention, there is provided a method for altering the phenotype of a tumor cell comprising the step of contacting the cell with a nucleic acid (i) encoding a tumor suppressor designated 86F and (ii) a promoter active in the tumor cell, wherein the promoter is operably linked to the region encoding the tumor suppressor, under conditions permitting the uptake of the nucleic acid by the tumor cell. The nucleic acid may be encapsulated in a liposome, or in a viral particle as part of a retrovirus, adenovirus, adeno-associated virus, vaccinia virus or herpesvirus.

In another embodiment, there is provided a method for treating a subject with cancer comprising the step of administering to the subject a tumor suppressor designated 86F. The tumor cell may be derived from a tissue selected from the group consisting of brain, lung, liver, spleen, kidney, lymph node, small intestine, blood cells, pancreas, colon, stomach, breast, endometrium, prostate, testicle, ovary, skin, head and neck, esophagus, bone marrow and blood tissue. The subject may be a human. The method may comprise the step of administering to the subject a nucleic acid (i) encoding a tumor suppressor designated 86F and (ii) a promoter active in eukaryotic cells, wherein the promoter is operably linked to the region encoding the tumor suppressor.

Also provided is a non-human transgenic eukaryote lacking a functional 86F gene. The eukaryote may be a mammal. The mammal may be a mouse. Another embodiment is a non-human transgenic eukaryotic that overexpresses 86F as compared to a similar non-transgenic eukaryote. Another embodiment is a non-human transgenic eukaryotic that overexpresses 86S as compared to a similar non-transgenic eukaryote. Another embodiment is a non-human transgenic eukaryote that overexpresses 86Y as compared to a similar non-transgenic eukaryote.

In yet another aspect of the invention, there is provided a method of screening a candidate substance for anti-tumor activity comprising the steps of (i) providing a cell lacking functional 86F polypeptide; (ii) contacting the cell with the candidate substance; and (iii) determining the effect of the candidate substance on the cell. The cell may be a tumor cell, for example, one that has a mutation in the coding region of 86F. The tumor cell may have aberrant methylation patterns in the regulatory or coding region of 86F, or be a deletion mutant, an insertion mutant, a frameshift mutant, a nonsense mutant, a missense mutant or splice mutant. The determining may comprise comparing one or more characteristics of the cell in the presence of the candidate substance with characteristics of a cell in the absence of the candidate substance. The characteristic may be 86F expression, 86S expression, 86Y expression, IGFBP2 expression, phosphatase activity, proliferation, metastasis, contact inhibition, soft agar growth, cell cycle regulation, tumor formation, tumor progression and tissue invasion. The candidate substance may be a chemotherapeutic, genetic or radiotherapeutic agent. The candidate substance also may be selected from a small molecule library. The cell may be contacted in vitro or in vivo.

In a further embodiment, there is provided an anti-tumor composition made according to the method comprising the steps of (i) providing a cell lacking functional 86F polypeptide; (ii) contacting the cell with the candidate substance; (iii) determining the effect of the candidate substance on the cell; (iv) identifying a candidate inhibitor substance; and (v) making the composition.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Protein alignment for 86F (86J), 86S and 86Y Polypeptides.

FIG. 2. Protein alignment for 86F (86J), 86S and 86Y Polynucleotides.

FIG. 3. Exonic structure of 86 variants.

### SEQUENCE SUMMARY

SEQ ID NO: 1=86F polypeptide; SEQ ID NO: 2=86S polypeptide; SEQ ID NO: 3=86Y polypeptide; SEQ ID NO: 4=86F polynucleotide; SEQ ID NO: 5=86S polynucleotide; SEQ ID NO: 6=86Y polynucleotide.

### DETAILED DESCRIPTION OF THE PREFERRED EMBODIMENTS

### I. THE PRESENT INVENTION

Glioma-glioblastoma multiforme (GBM) is a highly malignant neuroepithelial neoplasm, affecting both children, behaving similarly in both. However, it is more rapidly fatal in children than other similarly situated gliomas in childhood. Overall survival rate is very poor (only one-third of children affected are alive at one year after diagnosis), although chemotherapy and with surgery offer some hope in increasing survival time. Therefore, understanding the molecular mechanisms of the disease is critical to early diagnosis and improving the clinical outcome.

Using a cDNA microarray, the inventors previously demonstrated that IGFBP2 is overexpressed in most advanced stage GBM. Fuller et al., 1999. As discussed above, it also is implicated in number of other cancers. Using a two-hybrid screen, the inventors sought to identify other proteins that interact with IGFBP2. This study identified a protein that binds to IGFBP2, the gene for which is localized at chromosome 1p36, a region previously implicated in tumorigenesis by the loss of heterozygosity and by deletion, rearrangement, or underexpression in many cancer cell lines. This gene had previously been discovered as part of the cDNA clone sequenced by the Japanese Human cDNA Sequence project (Accession No. AK024020.1; SEQ. ID. NO. 7). However, this clone has an extra fragment of 182 base pairs inserted in the 3′ end. After recovering and sequencing the full-length cDNA, the inventors confirmed that this clone was an isoform of the same gene.

Structural analysis of the gene identified a domain that is 90% identical to the caspase activation domain, indicating that the gene likely possesses a tumor suppressor function. The inventors also identified a splice variant of the gene that has only been found in cancer cell lines. This discovery facilitates a wide range of endeavors including diagnosis, therapy, and drug screening.

### II. GLIOMA-GLIOBLASTOMA MULTIFORME

Glioma-glioblastoma multiforme (GBM) is the most malignant of the neuroepithelial neoplasms, characterized by cellular pleomorphism, numerous mitotic figures, and often multinucleated giant cell. Proliferation of the vascular endothelium is seen as well as areas of necrosis with circumjacent pseudopalisading of the neoplastic cells. It can appear as either a well-circumscribed globular mass or a more diffuse mass lesion. The cut surface reveals necrosis, fatty degeneration, and hemorrhage. Hemorrhages have been found in 40%, with necrosis in up to 52% of the cases. The tumor is usually solid, although cysts may be present. Rarely the tumor consists of a solitary cyst and mural nodule.

Grading according to degree of malignancy was first proposed in 1949. In this classification, astrocytomas and glioblastomas represent different grades of malignancy of the same tumor. Grade I tumors, typically slow growing, are characterized by most cells having normal characteristics, and few mitotic features. Endothelial proliferation is absent. Grade II tumors, previously designated “astroblastomas,” are characterized by an increased number of cells with polymorphic nuclei in mitoses. There is no clear line of demarcation from normal tissue. Grade III tumors represent anaplastic astrocytomas and Grade IV tumors represent the typical glioblastoma multiforme, characterized by cellular pleomorphism, vascular proliferation, mitoses, and multinucleated giant cells.

Glioblastoma multiforme constitutes approximately 7% of childhood intracranial neoplasms. The overall male to female ratio in children is 3:2. In adults, glioblastomas are noted most frequently in the frontal lobe with the temporal lobe second in frequency. Childhood glioblastomas of the cerebral hemispheres are also located most often in the frontal lobe; with the second most frequent site being the parietal lobe. Primary glioblastoma of the spinal cord in childhood is rare.

Glioblastoma multiforme in children appears to have two characteristic courses, each of which is related to the location of the tumor. Glioblastomas of the brainstem, a more primitive part of the central nervous system, occur at a younger age and have a shorter mean survival relative to those of the cerebral hemispheres. Glioblastoma multiforme of the cerebral hemisphere, a more highly developed part of the central nervous system, is characterized by onset in older children (13 years) and by a longer mean survival.

Headache is the most common complaint and papilledema the most common physical finding in children with hemispheric glioblastoma. Seizures are noted in up to one third of the children. Survival rates in patients with glioblastoma multiforme is uniformly poor. In studies of children treated with surgery and intracranial radiation, only one third of the children are alive one year after diagnosis. Survival of children with glioblastoma multiforme of either of the cerebral hemispheres or the brainstem has significantly increased since the advent of dexamethasone therapy. Presently therapy consists of surgery plus combination chemotherapy.

In summary it can be said that glioblastoma multiforme behaves similarly in both children and adults. The course of intracranial glioblastomas in children is more rapidly fatal than that of other similarly situated gliomas in childhood. While the overall survival rate is very poor in patients with a glioblastoma multiforme, intensive chemotherapy with surgical resection does offer some hope in increasing survival time among children.

### III. THE 86 TUMOR SUPPRESSOR

According to the present invention, the inventors have identified a tumor suppressor encoded by a gene in the 1p36 locus, and herein designated as “86.” The term tumor suppressor is well-known to those of skill in the art. Examples of other tumors suppressors are p53, Rb and p16, to name a few. While these molecules are structurally distinct, they form a group of functionally-related molecules, of which 86 is a member. The uses in which these other tumor suppressors now are being exploited are equally applicable here.

A. Features of the Polypeptide

The gene for 86 encodes either a 388 amino acid polypeptide (86F; SEQ ID NO: 1) or a 358 amino acid polypeptide (86S; SEQ ID NO: 2), depending on a splicing variation that eliminates exon 7 in the latter. A third molecule, 86Y (SEQ ID NO: 3) contains exon 7, like 86F, but also contains additional sequence in exon 9, lacking in both the other 86 proteins. When the present application refers to the function of 86 or “wild-type” activity, it is meant that the molecule in question has the ability to inhibit the transformation of a cell from a normally regulated state of proliferation to a malignant state, i.e., one associated with any sort of abnormal growth regulation, or to inhibit the transformation of a cell from an abnormal state to a highly malignant state, e.g., to prevent metastasis or invasive tumor growth. Other phenotypes that may be associated with normal 86 protein function are angiogenesis, adhesion, migration, cell-to-cell signaling, cell growth, cell proliferation, density-dependent growth, anchorage-dependent growth and others. Determination of which molecules possess this activity may be achieved using assays familiar to those of skill in the art. For example, transfer of genes encoding 86, or variants thereof, into cells that do not have a functional 86 product, and hence exhibit impaired growth control, will identify, by virtue of growth suppression, those molecules having 86 function.

B. Variants of 86

Amino acid sequence variants of the polypeptide can be substitutional, insertional or deletion variants. Deletion variants lack one or more residues of the native protein which are not essential for function or immunogenic activity. A common type of deletion variant is one lacking secretory signal sequences or signal sequences directing a protein to bind to a particular part of a cell. As discussed above, a natural variant of 86 is a splice variant that results in the deletion of 30 amino acids. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those that are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine*−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of 86, but with altered and even improved characteristics.

C. Peptides

The present invention also describes smaller, 86-related peptides for use in various embodiments of the present invention. In general, peptides are 50 amino acid residues or less, comprising contiguous residues of SEQ ID NOS: 1-3. In certain embodiments the size of the peptide is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 residues. Peptides may be produced from full length 86 protein by proteolytic digestion with enzymes such as trypsin or chymotrypsin.

Because of their relatively small size, the peptides of the invention can also be synthesized in solution or on a solid support in accordance with conventional techniques. Various automatic synthesizers are commercially available and can be used in accordance with known protocols. See, for example, Stewart and Young, (1984); Merrifield, (1986); and Barany and Merrifield (1979), each incorporated herein by reference. Short peptide sequences, or libraries of overlapping peptides, usually from about 6 up to about 50 amino acids, which correspond to selected regions of the 86 protein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

D. Domain Switching

Domain switching involves the generation of chimeric molecules using different but, in this case, related polypeptides. By comparing the 86 sequences, both with mutants and allelic variants, one can make predictions as to the functionally significant regions of these molecules. It is possible, then, to switch related domains of these molecules in an effort to determine the criticality of these regions to 86 function. These molecules may have additional value in that these “chimeras” can be distinguished from natural molecules, while possibly providing the same function.

E. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

F. Purification of Proteins

It will be desirable to purify 86 polypeptides or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are: ion-exchange chromatography, exclusion chromatography; polyacrylamide gel electrophoresis; and isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “−fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “−fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins. Other lectins that have been used include lentil lectin, wheat germ agglutinin, which has been useful in the purification of N-acetyl glucosaminyl residues, and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

G. Antigen Composition

The present invention also provides for the use of 86 proteins or peptides as antigens for the immunization of animals relating to the production of antibodies. It is envisioned that either 86, or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents for the carriers are keyhole limpet hemocyannin (KLH) or bovine serum albumin (BSA).

### IV. NUCLEIC ACIDS

The present invention also provides, in another embodiment, genes encoding 86. The genes for the human 86 molecule have been identified. The present invention is not limited in scope to these genes, however, as one of ordinary skill in the art could, using these nucleic acids, readily identify related homologs in various other species (e.g., mouse, rat, rabbit, dog, monkey, gibbon, chimp, ape, baboon, cow, pig, horse, sheep, cat and other species).

In addition, it should be clear that the present invention is not limited to the specific nucleic acids disclosed herein. As discussed below, an “86 gene” may contain a variety of different bases and still produce a corresponding polypeptide that is functionally, and in some cases structurally, indistinguishable from the human genes disclosed herein.

Similarly, any reference to a nucleic acid should be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance the function of 86.

A. Nucleic Acids Encoding 86

Nucleic acids according to the present invention may encode an entire 86 gene, a domain of 86 that expresses a tumor suppressing polypepride, or any other fragment of the 86 sequences set forth herein. The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a given 86 from a given species may be represented by natural variants that have slightly different nucleic acid sequences but, nonetheless, encode the same protein (see Table 1).

As used in this application, the term “a nucleic acid encoding an 86” refers to a nucleic acid molecule that has been isolated free of total cellular nucleic acid. In preferred embodiments, the invention concerns a nucleic acid sequence essentially as set forth in SEQ ID NOS: 4-6, a cDNA. The term “as set forth in SEQ ID NOS: 4-6” means that the nucleic acid sequence substantially corresponds to a portion of SEQ ID NOS: 4-6. The term “functionally equivalent codon” is used herein to refer to codons that encode the same amino acid, such as the six condons for arginine or serine (Table 1), and also refers to codons that encode biologically equivalent amino acids, as discussed in the following pages.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of SEQ ID NO: ([insert]). Sequences that are essentially the same as those set forth in SEQ ID NO: ([insert]) also may be functionally defined as sequences that are capable of hybridizing to a nucleic acid segment containing the complement of SEQ ID NO: ([insert]) under standard conditions.

The DNA segments of the present invention include those encoding biologically functional equivalent 86 proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

B. Oligonucleotide Probes and Primers

Naturally, the present invention also encompasses DNA segments that are complementary, or essentially complementary, to the sequence set forth in SEQ ID NOS: 4-6. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NOS: 4-6 under relatively stringent conditions such as those described herein. Such sequences may encode an entire 86 protein or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

One method of using probes and primers of the present invention is in the search for genes related to 86 or, more particularly, homologs of 86 from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single-stranded and double-stranded form. Typical vectors useful in site-directed mutagenesis include vectors such as the M13 phage. These phage vectors are commercially available and their use is generally well known to those skilled in the art. Double-stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double-stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. Antisense Constructs

In some cases, mutant tumor suppressors may not be non-functional. Rather, they may have aberrant functions that cannot be overcome by replacement gene therapy, even where the “wild-type” molecule is expressed in amounts in excess of the mutant polypeptide. Antisense treatments are one way of addressing this situation. Antisense technology also may be used to “knock-out” function of 86 in the development of cell lines or transgenic mice for research, diagnostic and screening purposes.

Antisense methodology takes advantage of the fact that nucleic acids tend to pair with “complementary” sequences. By complementary, it is meant that polynucleotides are those that are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

D. Ribozymes

Another approach for addressing the “dominant negative” mutant tumor suppressor is through the use of ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

E. Vectors for Cloning, Gene Transfer and Expression

Within certain embodiments expression vectors are employed to express the 86 polypeptide product, which can then be purified and, for example, be used to vaccinate animals to generate antisera or monoclonal antibody with which further studies may be conducted. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Maniatis et al., 1988 and Ausubel et al., 1994, both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

(i) Regulatory Elements

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the five non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Tables 2 lists several elements/promoters that may be employed, in the context of the present invention, to regulate the expression of a gene. This list is not intended to be exhaustive of all the possible elements involved in the promotion of expression but, merely, to be exemplary thereof. Table 3 provides examples of inducible elements, which are regions of a nucleic acid sequence that can be activated in response to a specific stimulus.

TABLE 2
Promoter and/or Enhancer

| Promoter/Enhancer | References |
|---|---|
| Immunoglobulin | Banerji et al., 1983; Gilles et al., 1983; |
| Heavy Chain | Grosschedl et al., 1985; Atchinson et al., |
| | 1986, 1987; Imler et al., 1987; Weinberger |
| | et al., 1984; Kiledjian et al., 1988; Porton |
| | et al.; 1990 |
| Immunoglobulin Light | Queen et al., 1983; Picard et al., 1984 |
| Chain | |
| T-Cell Receptor | Luria et al., 1987; Winoto et al., 1989; |
| | Redondo et al.; 1990 |
| HLA DQ a and/or | Sullivan et al., 1987 |
| DQ β | |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA- | Sherman et al., 1989 |
| Dra | |
| β-Actin | Kawamoto et al., 1988; Ng et al.; 1989 |
| Muscle Creatine | Jaynes et al., 1988; Horlick et al., 1989; |
| Kinase (MCK) | Johnson et al., 1989 |
| Prealbumin | Costa et al., 1988 |
| (Transthyretin) | |
| Elastase I | Ornitz et al., 1987 |
| Metallothionein | Karin et al., 1987; Culotta et al., 1989 |
| (MTII) | |
| Collagenase | Pinkert et al., 1987; Angel et al., 1987 |
| Albumin | Pinkert et al., 1987; Tronche et al., 1989, |
| | 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsch et al., 1990 |
| Molecule (NCAM) | |
| α₁-Antitrypsin | Latimer et al., 1990 |
| H2B (TH2B) Histone | Hwang et al., 1990 |
| Mouse and/or Type I | Ripe et al., 1989 |
| Collagen | |
| Glucose-Regulated | Chang et al., 1989 |
| Proteins (GRP94 and | |
| GRP78) | |
| Rat Growth Hormone | Larsen et al., 1986 |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor | |
| (PDGF) | |
| Duchenne Muscular | Klamut et al., 1990 |
| Dystrophy | |
| SV40 | Banerji et al., 1981; Moreau et al., 1981; |
| | Sleigh et al., 1985; Firak et al., 1986; Herr |
| | et al., 1986; Imbra et al., 1986; Kadesch et al., |
| | 1986; Wang et al., 1986; Ondek et al., 1987; Kuhl |
| | et al., 1987; Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi |
| | et al., 1988; Reisman et al., 1989 |
| Papilloma Virus | Campo et al., 1983; Lusky et al., 1983; |
| | Spandidos and/or Wilkie, 1983; Spalholz et al., |
| | 1985; Lusky et al., 1986; Cripe et al., 1987; |
| | Gloss et al., 1987; Hirochika et al., 1987; |
| | Stephens et al., 1987 |
| Hepatitis B Virus | Bulla et al., 1986; Jameel et al., 1986; Shaul |
| | et al., 1987; Spandau et al., 1988; Vannice et al., |
| | 1988 |
| Human Immuno- | Muesing et al., 1987; Hauber et al., 1988; |
| deficiency Virus | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus | Weber et al., 1984; Boshart et al., 1985; Foecking |
| (CMV) | et al., 1986 |
| Gibbon Ape Leukemia | Holbrook et al., 1987; Quinn et al., 1989 |
| Virus | |

TABLE 3
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa |
| | | et al., 1987, Karin et al., |
| | | 1987; Angel et al., 1987b; |
| | | McNeall et al., 1989 |
| MMTV (mouse | Glucocorticoids | Huang et al., 1981; Lee |
| mammary tumor | | et al., 1981; Majors et al., |
| virus) | | 1983; Chandler et al., |
| | | 1983; Lee et al., 1984; |
| | | Ponta et al., 1985; Sakai |
| | | et al., 1988 |
| β-Interferon | Poly(rI)x | Tavernier et al., 1983 |
| | Poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I | Interferon | Blanar et al., 1989 |
| Gene H-2κb | | |
| HSP70 | E1A, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor α | | |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), human platelet endothelial cell adhesion molecule-1 (Almendro et al., 1996).

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

(ii) IRES

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

(iii) Multi-Purpose Cloning Sites

Vectors can include a multiple cloning site (MCS), which is a nucleic acid region that contains multiple restriction enzyme sites, any of which can be used in conjunction with standard recombinant technology to digest the vector. See Carbonelli et al., 1999, Levenson et al., 1998, and Cocea, 1997, incorporated herein by reference. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

(iv) Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. See Chandler et al., 1997.

(v) Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

(vi) Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

(vii) Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

(viii) Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

(ix) Viral Vectors

The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simplex virus, retrovirus and adeno-associated virus vectors are being evaluated currently for treatment of diseases such as cancer, cystic fibrosis, Gaucher disease, renal disease and arthritis (Robbins and Ghivizzani, 1998; Imai et al., 1998; U.S. Pat. No. 5,670,488). The various viral vectors described below, present specific advantages and disadvantages, depending on the particular gene-therapeutic application.

Adenoviral Vectors: In particular embodiments, an adenoviral expression vector is contemplated for the delivery of expression constructs. “Adenovirus expression vector” is meant to include those constructs containing adenovirus sequences sufficient to (a) support packaging of the construct and (b) to ultimately express a tissue or cell-specific construct that has been cloned therein.

Adenoviruses comprise linear, double-stranded DNA, with a genome ranging from 30 to 35 kb in size (Reddy et al., 1998; Morrison et al., 1997; Chillon et al., 1999). An adenovirus expression vector according to the present invention comprises a genetically engineered form of the adenovirus. Advantages of adenoviral gene transfer include the ability to infect a wide variety of cell types, including non-dividing cells, a mid-sized genome, ease of manipulation, high infectivity and the ability to be grown to high titers (Wilson, 1996). Further, adenoviral infection of host cells does not result in chromosomal integration because adenoviral DNA can replicate in an episomal manner, without potential genotoxicity associated with other viral vectors. Adenoviruses also are structurally stable (Marienfeld et al., 1999) and no genome rearrangement has been detected after extensive amplification (Parks and Graham, 1997; Bett et al., 1993).

Salient features of the adenovirus genome are an early region (E1, E2, E3 and E4 genes), an intermediate region (pIX gene, Iva2 gene), a late region (L1, L2, L3, L4 and L5 genes), a major late promoter (MLP), inverted-terminal-repeats (ITRs) and a sequence (Zheng, et al., 1999; Robbins et al., 1998; Graham and Prevec, 1995). The early genes E1, E2, E3 and E4 are expressed from the virus after infection and encode polypeptides that regulate viral gene expression, cellular gene expression, viral replication, and inhibition of cellular apoptosis. Further on during viral infection, the MLP is activated, resulting in the expression of the late (L) genes, encoding polypeptides required for adenovirus encapsidation. The intermediate region encodes components of the adenoviral capsid. Adenoviral inverted terminal repeats (ITRs; 100-200 bp in length), are cis elements, and function as origins of replication and are necessary for viral DNA replication. The sequence is required for the packaging of the adenoviral genome.

A common approach for generating an adenoviruses for use as a gene transfer vector is the deletion of the E1 gene (E1⁻), which is involved in the induction of the E2, E3 and E4 promoters (Graham and Prevec, 1995). Subsequently, a therapeutic gene or genes can be inserted recombinantly in place of the E1 gene, wherein expression of the therapeutic gene(s) is driven by the E1 promoter or a heterologous promoter. The E1⁻, replication-deficient virus is then proliferated in a “helper” cell line that provides the E1 polypeptides in trans (e.g., the human embryonic kidney cell line 293). Thus, in the present invention it may be convenient to introduce the transforming construct at the position from which the E1-coding sequences have been removed. However, the position of insertion of the construct within the adenovirus sequences is not critical to the invention. Alternatively, the E3 region, portions of the E4 region or both may be deleted, wherein a heterologous nucleic acid sequence under the control of a promoter operable in eukaryotic cells is inserted into the adenovirus genome for use in gene transfer (U.S. Pat. Nos. 5,670,488 and 5,932,210, each specifically incorporated herein by reference).

Although adenovirus based vectors offer several unique advantages over other vector systems, they often are limited by vector immunogenicity, size constraints for insertion of recombinant genes and low levels of replication. The preparation of a recombinant adenovirus vector deleted of all open reading frames, comprising a full length dystrophin gene and the terminal repeats required for replication (Haecker et al., 1996) offers some potentially promising advantages to the above mentioned adenoviral shortcomings. The vector was grown to high titer with a helper virus in 293 cells and was capable of efficiently transducing dystrophin in mdx mice, in myotubes in vitro and muscle fibers in vivo. Helper-dependent viral vectors are discussed below.

A major concern in using adenoviral vectors is the generation of a replication-competent virus during vector production in a packaging cell line or during gene therapy treatment of an individual. The generation of a replication-competent virus could pose serious threat of an unintended viral infection and pathological consequences for the patient. Armentano et al., describe the preparation of a replication-defective adenovirus vector, claimed to eliminate the potential for the inadvertent generation of a replication-competent adenovirus (U.S. Pat. No. 5,824,544, specifically incorporated herein by reference). The replication-defective adenovirus method comprises a deleted E1 region and a relocated protein IX gene, wherein the vector expresses a heterologous, mammalian gene.

Other than the requirement that the adenovirus vector be replication defective, or at least conditionally defective, the nature of the adenovirus vector is not believed to be crucial to the successful practice of the invention. The adenovirus may be of any of the 42 different known serotypes and/or subgroups A-F. Adenovirus type 5 of subgroup C is the preferred starting material in order to obtain the conditional replication-defective adenovirus vector for use in the present invention. This is because adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Adenovirus growth and manipulation is known to those of skill in the art, and exhibits broad host range in vitro and in vivo (U.S. Pat. No. 5,670,488; U.S. Pat. No. 5,932,210; U.S. Pat. No. 5,824,544). This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. Many experiments, innovations, preclinical studies and clinical trials are currently under investigation for the use of adenoviruses as gene delivery vectors. For example, adenoviral gene delivery-based gene therapies are being developed for liver diseases (Han et al., 1999), psychiatric diseases (Lesch, 1999), neurological diseases (Hermens and Verhaagen, 1998), coronary diseases (Feldman et al., 1996), muscular diseases (Petrof, 1998), gastrointestinal diseases (Wu, 1998) and various cancers such as colorectal (Fujiwara and Tanaka, 1998), pancreatic, bladder (Irie et al., 1999), head and neck (Blackwell et al., 1999), breast (Stewart et al., 1999), lung (Batra et al., 1999) and ovarian (Vanderkwaak et al., 1999).

Retroviral Vectors: In certain embodiments of the invention, the use of retroviruses for gene delivery are contemplated. Retroviruses are RNA viruses comprising an RNA genome. When a host cell is infected by a retrovirus, the genomic RNA is reverse transcribed into a DNA intermediate which is integrated into the chromosomal DNA of infected cells. This integrated DNA intermediate is referred to as a provirus. A particular advantage of retroviruses is that they can stably infect dividing cells with a gene of interest (e.g., a therapeutic gene) by integrating into the host DNA, without expressing immunogenic viral proteins. Theoretically, the integrated retroviral vector will be maintained for the life of the infected host cell, expressing the gene of interest.

The retroviral genome and the proviral DNA have three genes: gag, pol, and env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication.

A recombinant retrovirus of the present invention may be genetically modified in such a way that some of the structural, infectious genes of the native virus have been removed and replaced instead with a nucleic acid sequence to be delivered to a target cell (U.S. Pat. Nos. 5,858,744 and 5,739,018, each incorporated herein by reference). After infection of a cell by the virus, the virus injects its nucleic acid into the cell and the retrovirus genetic material can integrate into the host cell genome. The transferred retrovirus genetic material is then transcribed and translated into proteins within the host cell. As with other viral vector systems, the generation of a replication-competent retrovirus during vector production or during therapy is a major concern. Retroviral vectors suitable for use in the present invention are generally defective retroviral vectors that are capable of infecting the target cell, reverse transcribing their RNA genomes, and integrating the reverse transcribed DNA into the target cell genome, but are incapable of replicating within the target cell to produce infectious retroviral particles (e.g., the retroviral genome transferred into the target cell is defective in gag, the gene encoding virion structural proteins, and/or in pol, the gene encoding reverse transcriptase). Thus, transcription of the provirus and assembly into infectious virus occurs in the presence of an appropriate helper virus or in a cell line containing appropriate sequences enabling encapsidation without coincident production of a contaminating helper virus.

The growth and maintenance of retroviruses is known in the art (U.S. Pat. Nos. 5,955,331 and 5,888,502, each specifically incorporated herein by reference). Nolan et al. describe the production of stable high titre, helper-free retrovirus comprising a heterologous gene (U.S. Pat. No. 5,830,725, specifically incorporated herein by reference). Methods for constructing packaging cell lines useful for the generation of helper-free recombinant retroviruses with amphoteric or ecotrophic host ranges, as well as methods of using the recombinant retroviruses to introduce a gene of interest into eukaryotic cells in vivo and in vitro are contemplated in the present invention (U.S. Pat. No. 5,955,331).

Currently, the majority of all clinical trials for vector-mediated gene delivery use murine leukemia virus (MLV)-based retroviral vector gene delivery (Robbins et al., 1998; Miller et al., 1993). Disadvantages of retroviral gene delivery includes a requirement for ongoing cell division for stable infection and a coding capacity that prevents the delivery of large genes. However, recent development of vectors such as lentivirus (e.g., HIV), simian immunodeficiency virus (SIV) and equine infectious-anemia virus (EIAV), which can infect certain non-dividing cells, potentially allow the in vivo use of retroviral vectors for gene therapy applications (Amado and Chen, 1999; Klimatcheva et al., 1999; Case et al., 1999). For example, HIV-based vectors have been used to infect non-dividing cells such as neurons (Miyatake et al., 1999), islets (Leibowitz et al., 1999) and muscle cells (Johnston et al., 1999). The therapeutic delivery of genes via retroviruses are currently being assessed for the treatment of various disorders such as inflammatory disease (Moldawer et al., 1999), AIDS (Amado et al., 1999; Engel and Kohn, 1999), cerebrovascular disease (Weihl et al., 1999) and hemophilia (Kay, 1998).

Herpesviral Vectors: Herpes simplex virus (HSV) type I and type II contain a double-stranded, linear DNA genome of approximately 150 kb, encoding 70-80 genes. Wild type HSV are able to infect cells lytically and to establish latency in certain cell types (e.g., neurons). Similar to adenovirus, HSV also can infect a variety of cell types including muscle (Yeung et al., 1999), ear (Derby et al., 1999), eye (Kaufman et al., 1999), tumors (Yoon et al., 1999; Howard et al., 1999), lung (Kohut et al., 1998), neuronal (Garrido et al., 1999; Lachmann and Efstathiou, 1999), liver (Kooby et al., 1999) and pancreatic islets (Rabinovitch et al., 1999).

HSV viral genes are transcribed by cellular RNA polymerase II and are temporally regulated, resulting in the transcription and subsequent synthesis of gene products in roughly three discemable phases or kinetic classes. These phases of genes are referred to as the Immediate Early (IE) or alpha genes, Early (E) or beta genes and Late (L) or gamma genes. Immediately following the arrival of the genome of a virus in the nucleus of a newly infected cell, the IE genes are transcribed. The efficient expression of these genes does not require prior viral protein synthesis. The products of IE genes are required to activate transcription and regulate the remainder of the viral genome.

For use in therapeutic gene delivery, HSV must be rendered replication-defective. Protocols for generating replication-defective HSV helper virus-free cell lines have been described (U.S. Pat. No. 5,879,934; U.S. Pat. No. 5,851,826, each specifically incorporated herein by reference in its entirety). One IE protein, ICP4, also known as alpha 4 or Vmw175, is absolutely required for both virus infectivity and the transition from IE to later transcription. Thus, due to its complex, multifunctional nature and central role in the regulation of HSV gene expression, ICP4 has typically been the target of HSV genetic studies.

Phenotypic studies of HSV viruses deleted of ICP4 indicate that such viruses will be potentially useful for gene transfer purposes (Krisky et al., 1998a). One property of viruses deleted for ICP4 that makes them desirable for gene transfer is that they only express the five other IE genes: ICP0, ICP6, ICP27, ICP22 and ICP47 (DeLuca et al., 1985), without the expression of viral genes encoding proteins that direct viral DNA synthesis, as well as the structural proteins of the virus. This property is desirable for minimizing possible deleterious effects on host cell metabolism or an immune response following gene transfer. Further deletion of IE genes ICP22 and ICP27, in addition to ICP4, substantially improve reduction of HSV cytotoxicity and prevented early and late viral gene expression (Krisky et al., 1998b).

The therapeutic potential of HSV in gene transfer has been demonstrated in various in vitro model systems and in vivo for diseases such as Parkinson's (Yamada et al., 1999), retinoblastoma, intracerebral and intradermal tumors (Moriuchi et al., 1998), B cell malignancies (Suzuki et al., 1998) and Duchenne muscular dystrophy (Huard et al., 1997).

Adeno-Associated Viral Vectors: Adeno-associated virus (AAV), a member of the parvovirus family, is a human virus that is increasingly being used for gene delivery therapeutics. AAV has several advantageous features not found in other viral systems. First, AAV can infect a wide range of host cells, including non-dividing cells. Second, AAV can infect cells from different species. Third, AAV has not been associated with any human or animal disease and does not appear to alter the biological properties of the host cell upon integration. For example, it is estimated that 80-85% of the human population has been exposed to AAV. Finally, AAV is stable at a wide range of physical and chemical conditions which lends itself to production, storage and transportation requirements.

The AAV genome is a linear, single-stranded DNA molecule containing 4681 nucleotides. The AAV genome generally comprises an internal non-repeating genome flanked on each end by inverted terminal repeats (ITRs) of approximately 145 bp in length. The ITRs have multiple functions, including origins of DNA replication, and as packaging signals for the viral genome. The internal non-repeated portion of the genome includes two large open reading frames, known as the AAV replication (rep) and capsid (cap) genes. The rep and cap genes code for viral proteins that allow the virus to replicate and package the viral genome into a virion. A family of at least four viral proteins are expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus requiring co-infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. Although AAV can infect cells from different species, the helper virus must be of the same species as the host cell (e.g., human AAV will replicate in canine cells co-infected with a canine adenovirus).

AAV has been engineered to deliver genes of interest by deleting the internal non-repeating portion of the AAV genome and inserting a heterologous gene between the ITRs. The heterologous gene may be functionally linked to a heterologous promoter (constitutive, cell-specific, or inducible) capable of driving gene expression in target cells. To produce infectious recombinant AAV (rAAV) containing a heterologous gene, a suitable producer cell line is transfected with a rAAV vector containing a heterologous gene. The producer cell is concurrently transfected with a second plasmid harboring the AAV rep and cap genes under the control of their respective endogenous promoters or heterologous promoters. Finally, the producer cell is infected with a helper virus.

Once these factors come together, the heterologous gene is replicated and packaged as though it were a wild-type AAV genome. When target cells are infected with the resulting rAAV virions, the heterologous gene enters and is expressed in the target cells. Because the target cells lack the rep and cap genes and the adenovirus helper genes, the rAAV cannot further replicate, package or form wild-type AAV.

The use of helper virus, however, presents a number of problems. First, the use of adenovirus in a rAAV production system causes the host cells to produce both rAAV and infectious adenovirus. The contaminating infectious adenovirus can be inactivated by heat treatment (56C. for 1 hour). Heat treatment, however, results in approximately a 50% drop in the titer of functional rAAV virions. Second, varying amounts of adenovirus proteins are present in these preparations. For example, approximately 50% or greater of the total protein obtained in such rAAV virion preparations is free adenovirus fiber protein. If not completely removed, these adenovirus proteins have the potential of eliciting an immune response from the patient. Third, AAV vector production methods which employ a helper virus require the use and manipulation of large amounts of high titer infectious helper virus, which presents a number of health and safety concerns, particularly in regard to the use of a herpesvirus. Fourth, concomitant production of helper virus particles in rAAV virion producing cells diverts large amounts of host cellular resources away from rAAV virion production, potentially resulting in lower rAAV virion yields.

Lentiviral Vectors: Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

Lentiviral vectors are known in the art, see Naldini et al., (1996); Zufferey et al., (1997); U.S. Pat. Nos. 6,013,516;and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene, such as the STAT-1 gene in this invention, into that packaging cell yields a producer cell which releases infectious viral particles carrying the foreign gene of interest. The env preferably is an amphotropic envelope protein which allows transduction of cells of human and other species.

One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

The vector providing the viral env nucleic acid sequence is associated operably with regulatory sequences, e.g., a promoter or enhancer. The regulatory sequence can be any eukaryotic promoter or enhancer, including for example, the Moloney murine leukemia virus promoter-enhancer element, the human cytomegalovirus enhancer or the vaccinia P7.5 promoter. In some cases, such as the Moloney murine leukemia virus promoter-enhancer element, the promoter-enhancer elements are located within or adjacent to the LTR sequences.

The heterologous or foreign nucleic acid sequence, such as the STAT-1 encoding polynucleotide sequence herein, is linked operably to a regulatory nucleic acid sequence. Preferably, the heterologous sequence is linked to a promoter, resulting in a chimeric gene. The heterologous nucleic acid sequence may also be under control of either the viral LTR promoter-enhancer signals or of an internal promoter, and retained signals within the retroviral LTR can still bring about efficient expression of the transgene. Marker genes may be utilized to assay for the presence of the vector, and thus, to confirm infection and integration. The presence of a marker gene ensures the selection and growth of only those host cells which express the inserts. Typical selection genes encode proteins that confer resistance to antibiotics and other toxic substances, e.g., histidinol, puromycin, hygromycin, neomycin, methotrexate, etc., and cell surface markers.

The vectors are introduced via transfection or infection into the packaging cell line. The packaging cell line produces viral particles that contain the vector genome. Methods for transfection or infection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with a dominant selectable marker, such as neo, DHFR, Gln synthetase or ADA, followed by selection in the presence of the appropriate drug and isolation of clones. The selectable marker gene can be linked physically to the packaging genes in the construct.

Lentiviral transfer vectors Naldini et al. (1996), have been used to infect human cells growth-arrested in vitro and to transduce neurons after direct injection into the brain of adult rats. The vector was efficient at transferring marker genes in vivo into the neurons and long term expression in the absence of detectable pathology was achieved. Animals analyzed ten months after a single injection of the vector showed no decrease in the average level of transgene expression and no sign of tissue pathology or immune reaction (Blomer et al., 1997). Thus, in the present invention, one may graft or transplant cells infected with the recombinant lentivirus ex vivo, or infect cells in vivo.

Other Viral Vectors: The development and utility of viral vectors for gene delivery is constantly improving and evolving. Other viral vectors such as poxvirus; e.g., vaccinia virus (Gnant et al., 1999; Gnant et al., 1999), alpha virus; e.g., sindbis virus, Semliki forest virus (Lundstrom, 1999), reovirus (Coffey et al., 1998) and influenza A virus (Neumann et al., 1999) are contemplated for use in the present invention and may be selected according to the requisite properties of the target system.

In certain embodiments, vaccinia viral vectors are contemplated for use in the present invention. Vaccinia virus is a particularly useful eukaryotic viral vector system for expressing heterologous genes. For example, when recombinant vaccinia virus is properly engineered, the proteins are synthesized, processed and transported to the plasma membrane. Vaccinia viruses as gene delivery vectors have recently been demonstrated to transfer genes to human tumor cells, e.g., EMAP-II (Gnant et al., 1999), inner ear (Derby et al., 1999), glioma cells, e.g., p53 (Timiryasova et al., 1999) and various mammalian cells, e.g., P-450 (U.S. Pat. No. 5,506,138). The preparation, growth and manipulation of vaccinia viruses are described in U.S. Pat. No. 5,849,304 and U.S. Pat. No. 5,506,138 (each specifically incorporated herein by reference).

In other embodiments, sindbis viral vectors are contemplated for use in gene delivery. Sindbis virus is a species of the alphavirus genus (Garoff and Li, 1998) which includes such important pathogens as Venezuelan, Western and Eastern equine encephalitis viruses (Sawai et al., 1999; Mastrangelo et al, 1999). In vitro, sindbis virus infects a variety of avian, mammalian, reptilian, and amphibian cells. The genome of sindbis virus consists of a single molecule of single-stranded RNA, 11,703 nucleotides in length. The genomic RNA is infectious, is capped at the 5′ terminus and polyadenylated at the 3′ terminus, and serves as mRNA. Translation of a vaccinia virus 26S mRNA produces a polyprotein that is cleaved co- and post-translationally by a combination of viral and presumably host-encoded proteases to give the three virus structural proteins, a capsid protein (C) and the two envelope glycoproteins (E1 and PE2, precursors of the virion E2).

Three features of sindbis virus suggest that it would be a useful vector for the expression of heterologous genes. First, its wide host range, both in nature and in the laboratory. Second, gene expression occurs in the cytoplasm of the host cell and is rapid and efficient. Third, temperature-sensitive mutations in RNA synthesis are available that may be used to modulate the expression of heterologous coding sequences by simply shifting cultures to the non-permissive temperature at various time after infection. The growth and maintenance of sindbis virus is known in the art (U.S. Pat. No. 5,217,879, specifically incorporated herein by reference).

Chimeric Viral Vectors: Chimeric or hybrid viral vectors are being developed for use in therapeutic gene delivery and are contemplated for use in the present invention. Chimeric poxviral/retroviral vectors (Holzer et al., 1999), adenoviral/retroviral vectors (Feng et al., 1997; Bilbao et al., 1997; Caplen et al., 1999) and adenoviral/adeno-associated viral vectors (U.S. Pat. No. 5,871,982) have been described.

These “chimeric” viral gene transfer systems can exploit the favorable features of two or more parent viral species. For example, Wilson et al., provide a chimeric vector construct which comprises a portion of an adenovirus, AAV 5′ and 3′ ITR sequences and a selected transgene, described below (U.S. Pat. No. 5,871,983, specifically incorporate herein by reference).

The adenovirus/AAV chimeric virus uses adenovirus nucleic acid sequences as a shuttle to deliver a recombinant AAV/transgene genome to a target cell. The adenovirus nucleic acid sequences employed in the hybrid vector can range from a minimum sequence amount, which requires the use of a helper virus to produce the hybrid virus particle, to only selected deletions of adenovirus genes, which deleted gene products can be supplied in the hybrid viral production process by a selected packaging cell. At a minimum, the adenovirus nucleic acid sequences employed in the pAdA shuttle vector are adenovirus genomic sequences from which all viral genes are deleted and which contain only those adenovirus sequences required for packaging adenoviral genomic DNA into a preformed capsid head. More specifically, the adenovirus sequences employed are the cis-acting 5′ and 3′ inverted terminal repeat (ITR) sequences of an adenovirus (which function as origins of replication) and the native 5′ packaging/enhancer domain, that contains sequences necessary for packaging linear Ad genomes and enhancer elements for the E1 promoter. The adenovirus sequences may be modified to contain desired deletions, substitutions, or mutations, provided that the desired function is not eliminated.

The AAV sequences useful in the above chimeric vector are the viral sequences from which the rep and cap polypeptide encoding sequences are deleted. More specifically, the AAV sequences employed are the cis-acting 5′ and 3′ inverted terminal repeat (ITR) sequences. These chimeras are characterized by high titer transgene delivery to a host cell and the ability to stably integrate the transgene into the host cell chromosome (U. S. Pat. No. 5,871,983, specifically incorporate herein by reference). In the hybrid vector construct, the AAV sequences are flanked by the selected adenovirus sequences discussed above. The 5′ and 3′ AAV ITR sequences themselves flank a selected transgene sequence and associated regulatory elements, described below. Thus, the sequence formed by the transgene and flanking 5′ and 3′ AAV sequences may be inserted at any deletion site in the adenovirus sequences of the vector. For example, the AAV sequences are desirably inserted at the site of the deleted E1a/E1b genes of the adenovirus. Alternatively, the AAV sequences may be inserted at an E3 deletion, E2a deletion, and so on. If only the adenovirus 5′ ITR/packaging sequences and 3′ ITR sequences are used in the hybrid virus, the AAV sequences are inserted between them.

The transgene sequence of the vector and recombinant virus can be a gene, a nucleic acid sequence or reverse transcript thereof, heterologous to the adenovirus sequence, which encodes a protein, polypeptide or peptide fragment of interest. The transgene is operatively linked to regulatory components in a manner which permits transgene transcription. The composition of the transgene sequence will depend upon the use to which the resulting hybrid vector will be put. For example, one type of transgene sequence includes a therapeutic gene which expresses a desired gene product in a host cell. These therapeutic genes or nucleic acid sequences typically encode products for administration and expression in a patient in vivo or ex vivo to replace or correct an inherited or non-inherited genetic defect or treat an epigenetic disorder or disease.

(x) Non-Viral Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

Injection: In certain embodiments, a nucleic acid may be delivered to an organelle, a cell, a tissue or an organism via one or more injections (i.e., a needle injection), such as, for example, either subcutaneously, intradermally, intramuscularly, intervenously or intraperitoneally. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985).

Electroporation: In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

Calcium Phosphate: In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

DEAE-Dextran: In another embodiment, a nucleic acid is delivered into a cell using DEAE-dextran followed by polyethylene glycol. In this manner, reporter plasmids were introduced into mouse myeloma and erythroleukemia cells (Gopal, 1985).

Sonication Loading: Additional embodiments of the present invention include the introduction of a nucleic acid by direct sonic loading. LTK-fibroblasts have been transfected with the thymidine kinase gene by sonication loading (Fechheimer et al., 1987).

Liposome-Mediated Transfection: In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

Receptor Mediated Transfection: Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0 273 085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

Microprojectile Bombardment: Microprojectile bombardment techniques can be used to introduce a nucleic acid into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

F. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAxBAC® 2.0 from INVITROGEN® and BACPACK™ BACULOVIRUS EXPRESSION SYSTEM FROM CLONTECH®.

Other examples of expression systems include STRATAGENE®'s COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to; gpt, that confers resistance to mycophenolic acid; neo, that confers resistance to the aminoglycoside G418; and hygro, that confers resistance to hygromycin.

G. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5, JM109, and KC8, as well as a number of commercially available bacterial hosts such as SURE® Competent Cells and SOLOPACK Gold Cells (STRATAGENE®, La Jolla). Alternatively, bacterial cells such as E. Coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

H. Cell Propagation

Animal cells can be propagated in vitro in two modes: as non-anchorage dependent cells growing in suspension throughout the bulk of the culture or as anchorage-dependent cells requiring attachment to a solid substrate for their propagation (i.e., a monolayer type of cell growth). Non-anchorage dependent or suspension cultures from continuous established cell lines are the most widely used means of large scale production of cells and cell products. However, suspension cultured cells have limitations, such as tumorigenic potential and lower protein production than adherent T-cells.

Large scale suspension culture of mammalian cells in stirred tanks is a common method for production of recombinant proteins. Two suspension culture reactor designs are in wide use—the stirred reactor and the airlift reactor. The stirred design has successfully been used on an 8000 liter capacity for the production of interferon. Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcomer section of the reactor. The main advantage of this design is the simplicity and lack of need for mechanical mixing. Typically, the height-to-diameter ratio is 10:1. The airlift reactor scales up relatively easily, has good mass transfer of gases and generates relatively low shear forces.

The antibodies of the present invention are particularly useful for the isolation of antigens by immunoprecipitation. Immunoprecipitation involves the separation of the target antigen component from a complex mixture, and is used to discriminate or isolate minute amounts of protein. For the isolation of membrane proteins cells must be solubilized into detergent micelles. Nonionic salts are preferred, since other agents such as bile salts, precipitate at acid pH or in the presence of bivalent cations. Antibodies are and their uses are discussed further, below.

### V. GENERATING ANTIBODIES REACTIVE WITH 86

In another aspect, the present invention contemplates an antibody that is immunoreactive with an 86 polypeptide of the present invention, or any portion thereof. An antibody can be a polyclonal or a monoclonal antibody. In a preferred embodiment, an antibody is a monoclonal antibody. The present inventors have made two antibodies, designated W86.46 and W86.116, that were raised against residues 48-62 (TPSTPETSSTSL) and residues 116-130 (SLGTSSLRDPEPSGR), respectively.

Means for preparing and characterizing antibodies are well known in the art. Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to 86-related antigen epitopes. Additionally, it is proposed that monoclonal antibodies specific to the particular 86 of different species may be utilized in other useful applications

In general, both polyclonal and monoclonal antibodies against 86 may be used in a variety of embodiments. For example, they may be employed in antibody cloning protocols to obtain cDNAs or genes encoding other 86. They may also be used in inhibition studies to analyze the effects of 86 related peptides in cells or animals. Anti-86 antibodies will also be useful in immunolocalization studies to analyze the distribution of 86 during various cellular events, for example, to determine the cellular or tissue-specific distribution of 86 polypeptides under different points in the cell cycle. A particularly useful application of such antibodies is in purifying native or recombinant 86, for example, using an antibody affinity column. The operation of all such immunological techniques will be known to those of skill in the art in light of the present disclosure.

Means for preparing and characterizing antibodies are well known in the art (see, e.g., Harlow and Lane, 1988; incorporated herein by reference). More specific examples of monoclonal antibody preparation are give in the examples below.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified 86 protein, polypeptide or peptide or cell expressing high levels of 86. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from mycloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

### VI. DIAGNOSING CANCERS INVOLVING 86

The 86 polypeptides and the corresponding genes may be employed as a diagnostic or prognostic indicator of cancer. More specifically, point mutations, deletions, insertions, or regulatory pertubations relating to 86 may cause cancer or promote cancer development, cause or promote tumor progression at a primary site, and/or cause or promote metastasis. In particular, the splice variation given rise to the short 86S version may prove useful in identifying cancer-causing molecular events.

A. Genetic Diagnosis

One embodiment of the instant invention comprises a method for detecting variation in the expression of 86. This may comprises determining that level of 86 or determining specific alterations in the expressed product. Obviously, this sort of assay has importance in the diagnosis of related cancers. Such cancer may involve cancers of the brain (glioblastomas, medulloblastoma, astrocytoma, oligodendroglioma, ependymomas), lung, liver, spleen, kidney, pancreas, small intestine, blood cells, lymph node, colon, breast, endometrium, stomach, prostate, testicle, ovary, skin, head and neck, esophagus, bone marrow, blood or other tissue. In particular, the present invention relates to the diagnosis of gliomas.

The biological sample can be any tissue or fluid. Various embodiments include cells of the skin, muscle, facia, brain, prostate, breast, endometrium, lung, head & neck, pancreas, small intestine, blood cells, liver, testes, ovaries, colon, skin, stomach, esophagus, spleen, lymph node, bone marrow or kidney. Other embodiments include fluid samples such as peripheral blood, lymph fluid, ascites, serous fluid, pleural effusion, sputum, cerebrospinal fluid, lacrimal fluid, stool or urine.

Nucleic acid used is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given patient with a statistically significant reference group of normal patients and patients that have 86-related pathologies. In this way, it is possible to correlate the amount or kind of 86 detected with various clinical states.

“Alterations” in 86 may include deletions, insertions, point mutations and duplications. Point mutations result in stop codons, frameshift mutations or amino acid substitutions. Somatic mutations are those occurring in non-germline tissues. Germ-line tissue can occur in any tissue and are inherited. Mutations in and outside the coding region also may affect the amount of 86 produced, both by altering the transcription of the gene or in destabilizing or otherwise altering the processing of either the transcript (mRNA) or protein.

A cell takes a genetic step toward oncogenic transformation when one allele of a tumor suppressor gene is inactivated due to inheritance of a germline lesion or acquisition of a somatic mutation. The inactivation of the other allele of the gene usually involves a somatic micromutation or chromosomal allelic deletion that results in loss of heterozygosity (LOH). Alternatively, both copies of a tumor suppressor gene may be lost by homozygous deletion.

It is contemplated that other mutations in the 86 gene may be identified in accordance with the present invention. A variety of different assays are contemplated in this regard, including but not limited to, fluorescent in situ hybridization (FISH), direct DNA sequencing, PFGE analysis, Southern or Northern blotting, single-stranded conformation analysis (SSCA), RNAse protection assay, allele-specific oligonucleotide (ASO), dot blot analysis, denaturing gradient gel electrophoresis, RFLP and PCR-SSCP.

(i) Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

In preferred embodiments, the probes or primers are labeled with radioactive species (³²P, ¹⁴C, ³⁵S, ³H, or other label), with a fluorophore (rhodamine, fluorescein) or a chemillumiscent (luciferase).

(ii) Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention, Walker et al., (1992).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double-stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single-stranded DNA, which is then converted to double stranded DNA, and then transcribed once again with an RNA polymerase such as T7 or SP6. The resulting products, whether truncated or complete, indicate target specific sequences.

Davey et al., EPA 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, M. A., In: PCR PROTOCOLS: A GUIDE TO METHODS AND APPLICATIONS, Academic Press, N.Y., 1990; Ohara et al., 1989; each herein incorporated by reference in their entirety).

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989), incorporated herein by reference in its entirety.

(iii) Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

(iv) Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

(v) Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al., 1989. For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences throughout the 86 gene that may then be analyzed by direct sequencing.

(vi) Kit Components

All the essential materials and reagents required for detecting and sequencing 86 and variants thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

(vii) Design and Theoretical Considerations for Relative Quantitative RT-PCR

Reverse transcription (RT) of RNA to cDNA followed by relative quantitative PCR (RT-PCR) can be used to determine the relative concentrations of specific mRNA species isolated from patients. By determining that the concentration of a specific mRNA species varies, it is shown that the gene encoding the specific mRNA species is differentially expressed.

In PCR, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

The second condition that must be met for an RT-PCR experiment to successfully determine the relative abundances of a particular mRNA species is that relative concentrations of the amplifiable cDNAs must be normalized to some independent standard. The goal of an RT-PCR experiment is to determine the abundance of a particular mRNA species relative to the average abundance of all mRNA species in the sample. In the experiments described below, mRNAs for β-actin, asparagine synthetase and lipocortin II were used as external and internal standards to which the relative abundance of other mRNAs are compared.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR assay for clinically derived materials. The problems inherent in clinical samples are that they are of variable quantity (making normalization problematic), and that they are of variable quality (necessitating the co-amplification of a reliable internal control, preferably of larger size than the target). Both of these problems are overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies may be performed using a more conventional relative quantitative RT-PCR assay with an external standard protocol. These assays sample the PCR products in the linear portion of their amplification curves. The number of PCR cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR assays can be superior to those derived from the relative quantitative RT-PCR assay with an internal standard.

One reason for this advantage is that without the internal standard/competitor, all of the reagents can be converted into a single PCR product in the linear range of the amplification curve, thus increasing the sensitivity of the assay. Another reason is that with only one PCR product, display of the product on an electrophoretic gel or another display method becomes less complex, has less background and is easier to interpret.

(viii) Chip Technologies

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al. (1994); Fodor et al. (1991).

B. Immunodiagnosis

Antibodies of the present invention can be used in characterizing the 86 content of healthy and diseased tissues, through techniques such as ELISAs and Western blotting. This may provide a screen for the presence or absence of malignancy or as a predictor of future cancer.

The use of antibodies of the present invention, in an ELISA assay is contemplated. For example, anti-86 antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for 86 that differs the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

### VII. METHODS OF THERAPIES

The present invention also involves, in another embodiment, the treatment of cancer. The types of cancer that may be treated, according to the present invention, is limited only by the involvement of 86. By involvement, it is not even a requirement that 86 be mutated or abnormal—the overexpression of this tumor suppressor may actually overcome other lesions within the cell. Thus, it is contemplated that a wide variety of tumors may be treated using 86 therapy, including cancers of the brain, lung, liver, spleen, kidney, lymph node, pancreas, small intestine, blood cells, colon, stomach, breast, endometrium, prostate, testicle, ovary, skin, head and neck, esophagus, bone marrow, blood or other tissue. In particular, carcinomas and neuroblastomas are contemplated for treatment.

In many contexts, it is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

A. Genetic Based Therapies

One of the therapeutic embodiments contemplated by the present inventors is the intervention, at the molecular level, in the events involved in the tumorigenesis of some cancers. Specifically, the present inventors intend to provide, to a cancer cell, an expression construct capable of providing 86 to that cell. The lengthy discussion of expression vectors and the genetic elements employed therein is incorporated into this section by reference. Particularly preferred expression vectors are viral vectors such as adenovirus, adeno-associated virus, herpesvirus, vaccinia virus and retrovirus. Also preferred is liposomally-encapsulated expression vector.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for various tumor types. The section below on routes contains an extensive list of possible routes. For practically any tumor, systemic delivery is contemplated. This will prove especially important for attacking microscopic or metastatic cancer. Where discrete tumor mass may be identified, a variety of direct (intratumoral), local and regional approaches may be taken. For example, the tumor may be directly injected with the expression vector. A tumor bed may be treated prior to, during or after resection. Following resection, one generally will deliver the vector by a catheter left in place following surgery. One may utilize the tumor vasculature to introduce the vector into the tumor by injecting a supporting vein or artery. A more distal blood supply route also may be utilized.

In a different embodiment, ex vivo gene therapy is contemplated. This approach is particularly suited, although not limited, to treatment of bone marrow associated cancers. In an ex vivo embodiment, cells from the patient are removed and maintained outside the body for at least some period of time. During this period, a therapy is delivered, after which the cells are reintroduced into the patient; hopefully, any tumor cells in the sample have been killed.

Autologous bone marrow transplant (ABMT) is an example of ex vivo gene therapy. Basically, the notion behind ABMT is that the patient will serve as his or her own bone marrow donor. Thus, a normally lethal dose of irradiation or chemotherapeutic may be delivered to the patient to kill tumor cells, and the bone marrow repopulated with the patients own cells that have been maintained (and perhaps expanded) ex vivo. Because, bone marrow often is contaminated with tumor cells, it is desirable to purge the bone marrow of these cells. Use of gene therapy to accomplish this goal is yet another way 86 may be utilized according to the present invention.

B. Immunotherapies

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

According to the present invention, it is unlikely that 86 could serve as a target for an immune effector given that (i) it is unlikely to be expressed on the surface of the cell and (ii) that the presence, not absence, of 86 is associated with the normal state. However, it is possible that particular mutant forms of 86 may be targeted by immunotherapy, either using antibodies, antibody conjugates or immune effector cells.

A more likely scenario is that immunotherapy could be used as part of a combined therapy, in conjunction with 86-targeted gene therapy. The general approach for combined therapy is discussed below. Generally, the tumor cell must bear some marker that is amenable to targeting, i.e., is not present on the majority of other cells. Many tumor marker exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

C. Protein Therapy

Another therapy approach is the provision, to a subject, of 86 polypeptide, active fragments, synthetic peptides, mimetics or other analogs thereof. The protein may be produced by recombinant expression means or, if small enough, generated by an automated peptide synthesizer. Formulations would be selected based on the route of administration and purpose including, but not limited to, liposomal formulations and classic pharmaceutical preparations.

D. Combined Therapy

Tumor cell resistance to DNA damaging agents represents a major problem in clinical oncology. One goal of current cancer research is to find ways to improve the efficacy of chemo- and radiotherapy. One way is by combining such traditional therapies with gene therapy. For example, the herpes simplex-thymidine kinase (HS-tk) gene, when delivered to brain tumors by a retroviral vector system, successfully induced susceptibility to the antiviral agent ganciclovir (Culver et al., 1992). In the context of the present invention, it is contemplated that 86 replacement therapy could be used similarly in conjunction with chemo- or radiotherapeutic intervention. It also may prove effective to combine 86 gene therapy with immunotherapy, as described above.

To kill cells, inhibit cell growth, inhibit metastasis, inhibit angiogenesis or otherwise reverse or reduce the malignant phenotype of tumor cells, using the methods and compositions of the present invention, one would generally contact a “target” cell with a 86 expression construct and at least one other agent. These compositions would be provided in a combined amount effective to kill or inhibit proliferation of the cell. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the agent.

Alternatively, the gene therapy treatment may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either 86 or the other agent will be desired. Various combinations may be employed, where the 86 protein or nucleic acid is “A” and the other agent is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are contemplated. Again, to achieve cell killing, both agents are delivered to a cell in a combined amount effective to kill the cell.

Agents or factors suitable for use in a combined therapy are any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide. In certain embodiments, the use of cisplatin in combination with a 86 expression construct is particularly preferred as this compound.

In treating cancer according to the invention, one would contact the tumor cells with an agent in addition to the expression construct. This may be achieved by irradiating the localized tumor site with radiation such as X-rays, UV-light, γ-rays or even microwaves. Alternatively, the tumor cells may be contacted with the agent by administering to the subject a therapeutically effective amount of a pharmaceutical composition comprising a compound such as, adriamycin, 5-fluorouracil, etoposide, camptothecin, actinomycin-D, mitomycin C, or more preferably, cisplatin. The agent may be prepared and used as a combined therapeutic composition, or kit, by combining it with a 86 expression construct, as described above.

Agents that directly cross-link nucleic acids, specifically DNA, are envisaged to facilitate DNA damage leading to a synergistic, antineoplastic combination with 86. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that the regional delivery of 86 expression constructs to patients with 86-linked cancers will be a very efficient method for delivering a therapeutically effective gene to counteract the clinical disease. Similarly, the chemo- or radiotherapy may be directed to a particular, affected region of the subjects body. Alternatively, systemic delivery of expression construct and/or the agent may be appropriate in certain circumstances, for example, where extensive metastasis has occurred.

In addition to combining 86-targeted therapies with chemo- and radiotherapies, it also is contemplated that combination with other gene therapies will be advantageous. For example, targeting of 86 and p53 or p16 mutations at the same time may produce an improved anti-cancer treatment. Any other tumor-related gene conceivably can be targeted in this manner, for example, p21, Rb, APC, DCC, NF-1, NF-2, BCRA2, p16, FHIT, WT-1, MEN-I, MEN-II, BRCA1, VHL, FCC, MCC, ras, myc, neu, raf, erb, src, fms, jun, trk, ret, gsp, hst, bcl and abl.

It also should be pointed out that any of the foregoing therapies may prove useful by themselves in treating a 86. In this regard, reference to chemotherapeutics and non-86 gene therapy in combination should also be read as a contemplation that these approaches may be employed separately.

E. Formulations and Routes for Administration to Patients

Where clinical applications are contemplated, it will be necessary to prepare pharmaceutical compositions—expression vectors, virus stocks, proteins, antibodies and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra. Of particular interest is direct intratumoral administration, perfusion of a tumor, or admininstration local or regional to a tumor, for example, in the local or regional vasculature or lymphatic system.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VIII. SCREENING OF MODULATORS OF 86 FUNCTION

The present invention also contemplates the use of 86 and active fragments, and nucleic acids coding therefor, in the screening of compounds for activity in either stimulating 86 activity, overcoming the lack of 86 or blocking the effect of a mutant 86 molecule. These assays may make use of a variety of different formats and may depend on the kind of “activity” for which the screen is being conducted. Contemplated functional “read-outs” include binding to a compound, inhibition of binding to a substrate, ligand, receptor or other binding partner by a compound, inhibition or stimulation of cell-to-cell signaling, growth, metastasis, cell division, cell migration, soft agar colony formation, contact inhibition, invasiveness, angiogenesis, apoptosis, tumor progression or other malignant phenotype.

A. In Vitro Assays

In one embodiment, the invention is to be applied for the screening of compounds that bind to the 86 molecule or fragment thereof. The polypeptide or fragment may be either free in solution, fixed to a support, expressed in or on the surface of a cell. Either the polypeptide or the compound may be labeled, thereby permitting determining of binding.

In another embodiment, the assay may measure the inhibition of binding of 86 to a natural or artificial substrate or binding partner. Competitive binding assays can be performed in which one of the agents (86, binding partner or compound) is labeled. Usually, the polypeptide will be the labeled species. One may measure the amount of free label versus bound label to determine binding or inhibition of binding. In particular, the inventors contemplate IGFBP2 as a binding parter in this assay.

Another technique for high throughput screening of compounds is described in WO 84/03564. Large numbers of small peptide test compounds are synthesized on a solid substrate, such as plastic pins or some other surface. The peptide test compounds are reacted with 86 and washed. Bound polypeptide is detected by various methods.

Purified 86 can be coated directly onto plates for use in the aforementioned drug screening techniques. However, antibodies to the polypeptide can be used to immobilize the polypeptide to a solid phase. Also, fusion proteins containing a reactive region (preferably a terminal region) may be used to link the 86 active region to a solid phase.

Various cell lines containing wild-type or natural or engineered mutations in 86 can be used to study various functional attributes of 86 and how a candidate compound affects these attributes. Methods for engineering mutations are described elsewhere in this document, as are naturally-occurring mutations in 86 that lead to, contribute to and/or otherwise cause malignancy. In such assays, the compound would be formulated appropriately, given its biochemical nature, and contacted with a target cell. Depending on the assay, culture may be required. The cell may then be examined by virtue of a number of different physiologic assays. Alternatively, molecular analysis may be performed in which the function of 86, or related pathways, may be explored. This may involve assays such as those for protein expression, enzyme function, substrate utilization, phosphorylation states of various molecules including 86, cAMP levels, mRNA expression (including differential display of whole cell or polyA RNA) and others.

B. In Vivo Assays

The present invention also encompasses the use of various animal models. Here, the identity seen between human and mouse 86 provides an excellent opportunity to examine the function of 86 in a whole animal system where it is normally expressed. By developing or isolating mutant cells lines that fail to express normal 86, one can generate cancer models in mice that will be highly predictive of cancers in humans and other mammals. These models may employ the orthotopic or systemic administration of tumor cells to mimic primary and/or metastatic cancers. Alternatively, one may induce cancers in animals by providing agents known to be responsible for certain events associated with malignant transformation and/or tumor progression. Finally, transgenic animals (discussed below) that lack a functional wild-type 86 protein may be utilized as models for cancer development and treatment.

Treatment of animals with test compounds will involve the administration of the compound, in an appropriate form, to the animal. Administration will be by any route the could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, survival, reduction of tumor burden or mass, arrest or slowing of tumor progression, elimination of tumors, inhibition or prevention of metastasis, increased activity level, improvement in immune effector function and improved food intake.

C. Rational Drug Design

The goal of rational drug design is to produce structural analogs of biologically active polypeptides or compounds with which they interact (agonists, antagonists, inhibitors, binding partners, etc. ). By creating such analogs, it is possible to fashion drugs which are more active or stable than the natural molecules, which have different susceptibility to alteration or which may affect the function of various other molecules. In one approach, one would generate a three-dimensional structure for 86 or a fragment thereof. This could be accomplished by x-ray crystallograph, computer modeling or by a combination of both approaches. An alternative approach, “alanine scan,” involves the random replacement of residues throughout molecule with alanine, and the resulting affect on function determined.

It also is possible to isolate a 86-specific antibody, selected by a functional assay, and then solve its crystal structure. In principle, this approach yields a pharmacore upon which subsequent drug design can be based. It is possible to bypass protein crystallograph altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have improved 86 activity or which act as stimulators, inhibitors, agonists, antagonists or 86 or molecules affected by 86 function. By virtue of the availability of cloned 86 sequences, sufficient amounts of 86 can be produced to perform crystallographic studies. In addition, knowledge of the polypeptide sequences permits computer employed predictions of structure-function relationships.

### IX. TRANSGENICS

In one embodiment of the invention, transgenic animals are produced which contain a functional transgene encoding a functional 86 polypeptide or variants thereof. Transgenic animals expressing 86 transgenes, recombinant cell lines derived from such animals and transgenic embryos may be useful in methods for screening for and identifying agents that induce or repress function of 86. Transgenic animals of the present invention also can be used as models for studying indications such as cancers.

In one embodiment of the invention, a 86 transgene is introduced into a non-human host to produce a transgenic animal expressing a human or murine 86 gene. The transgenic animal is produced by the integration of the transgene into the genome in a manner that permits the expression of the transgene. Methods for producing transgenic animals are generally described by Wagner and Hoppe (U.S. Pat. No. 4,873,191; which is incorporated herein by reference), Brinster et al. 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

It may be desirable to replace the endogenous 86 by homologous recombination between the transgene and the endogenous gene; or the endogenous gene may be eliminated by deletion as in the preparation of “knock-out” animals. Typically, a 86 gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which overexpress 86 or express a mutant form of the polypeptide. Alternatively, the absence of one or both alleles of a 86 gene in “knock-out” mice permits the study of the effects that a reduction in or loss of 86 protein has on a cell in vivo. Knock-out mice also provide a model for the development of 86-related cancers.

As noted above, transgenic animals and cell lines derived from such animals may find use in certain testing experiments. In this regard, transgenic animals and cell lines capable of expressing wild-type or mutant 86 may be exposed to test substances. These test substances can be screened for the ability to enhance wild-type 86 expression and or function or impair the expression or function of mutant 86.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification of an IGFB2 Binding Molecule

To identify potential proteins that interact with IGFBP2, which is overexpressed in a number of cancers, the inventors applied a yeast two-hybrid system to screen a human fetal brain cDNA library. After three levels of selection, a few positive clones were isolated that interact with IGFBP2. The positive clones grew on Ade His media and turned blue when X-gal was supplied in the media. One of these clones was tentatively designated as clone 86.

To determine if the protein-protein interaction of IGFBP2 and clone 86 protein occurred in mammalian cells, the inventors performed co-immunoprecipitation using HEK 293 cells. The results showed that IGFBP2 was able to pull down the clone 86 protein, suggesting that this interaction indeed occurred in the mammalian cells, and was not merely an artifact of the two-hybrid system.

### Example 2

### Analysis of the 86 Gene and Protein

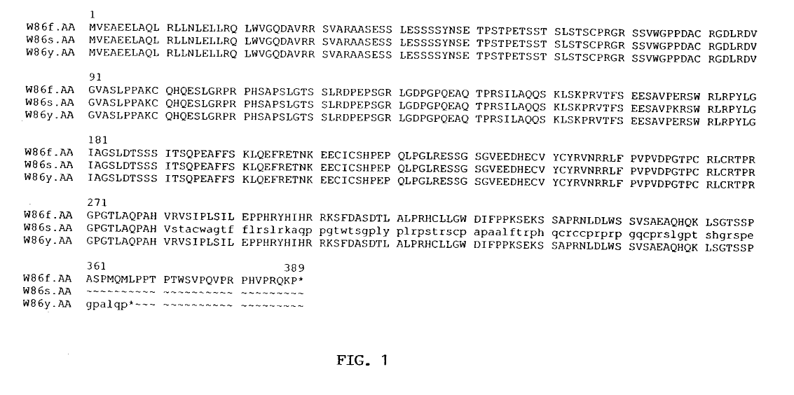

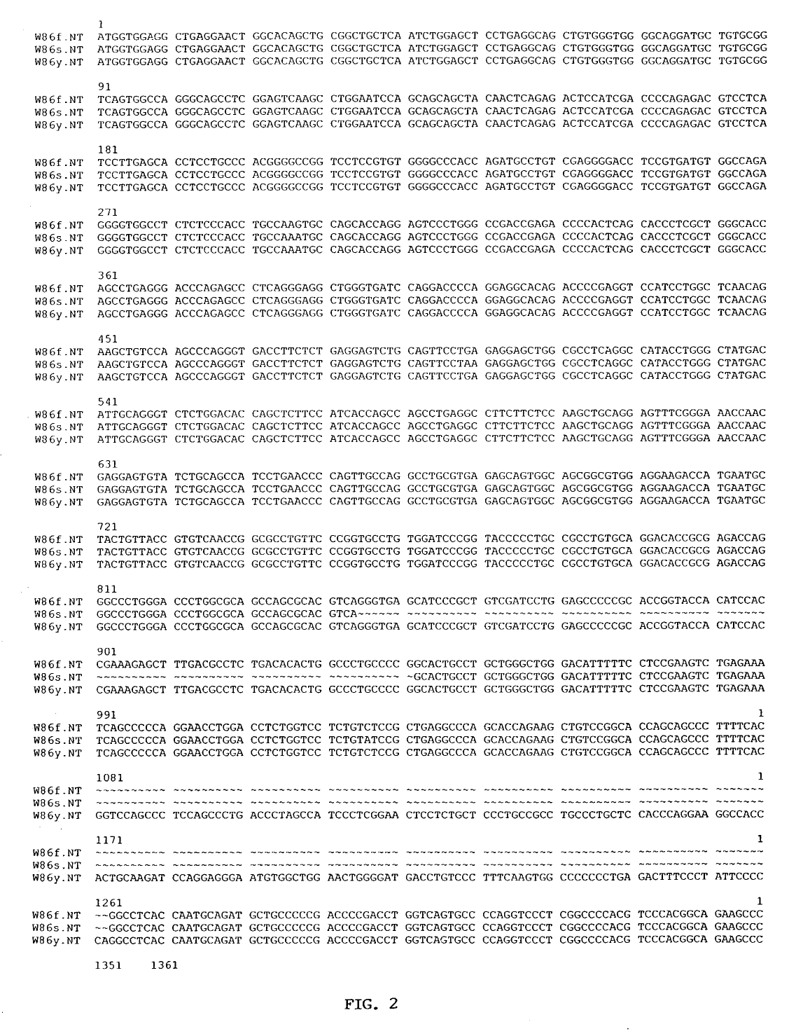

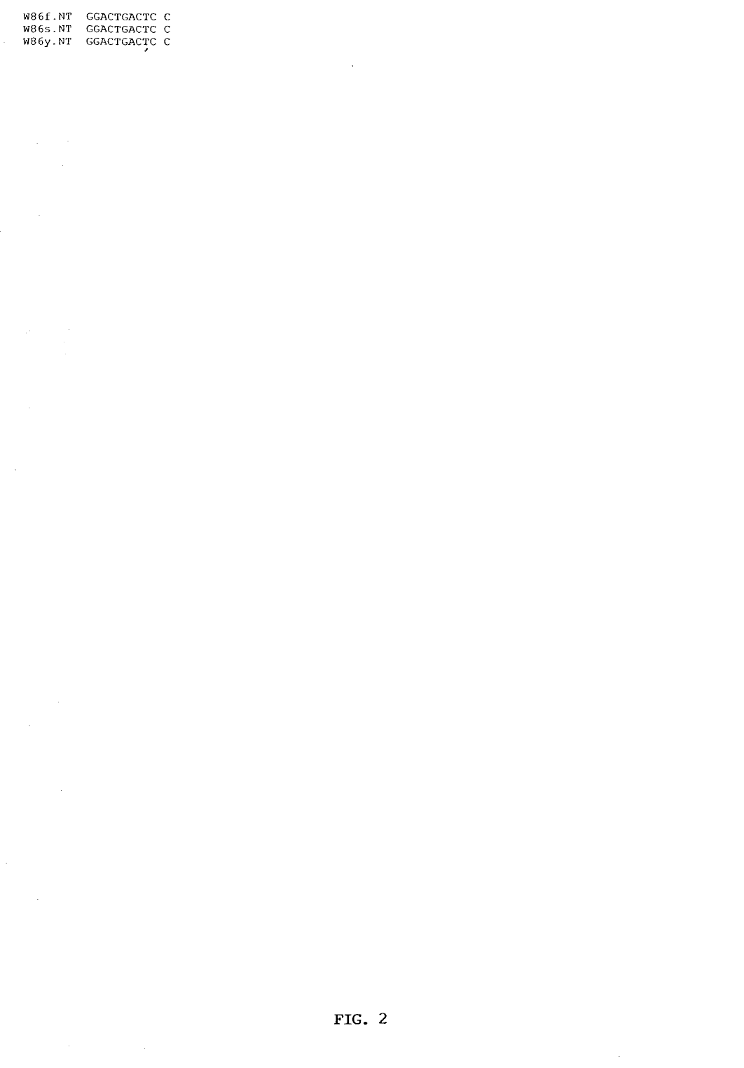

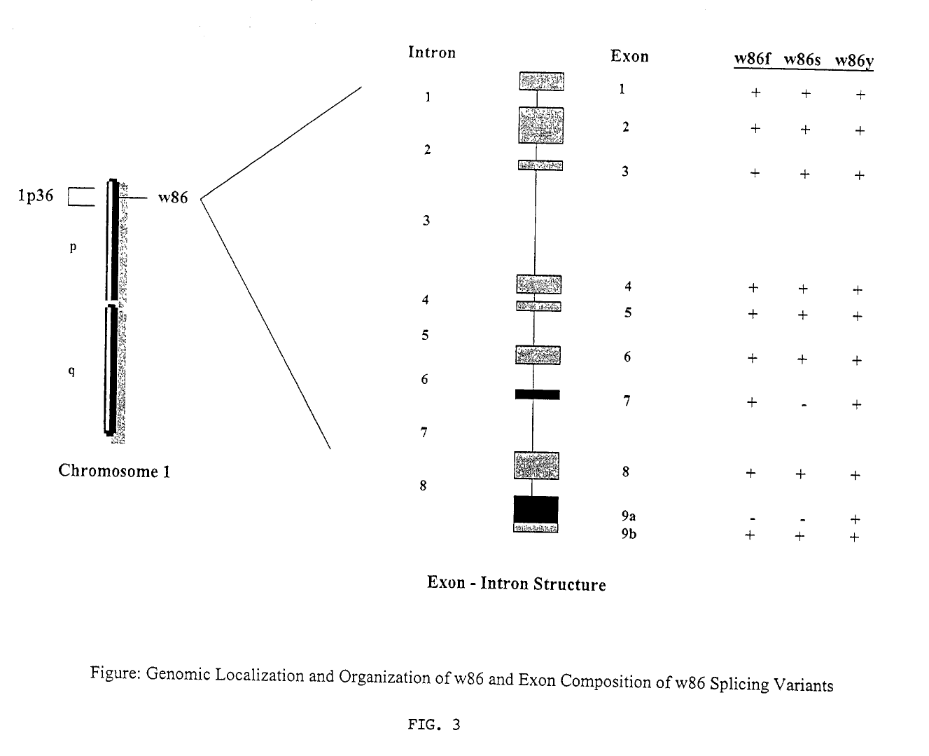

Sequencing analysis of clone 86 showed that it was not a full-length cDNA because it lacked a 5′-ATG starting site. This original this clone shared 100% identity to the 86J cDNA in nucleotide sequence, but lacked a 5′ segment of 234 bps, and contains an insertion of 172 bps near the 3′-end. 86J is an unknown gene submitted as part of the Japanese Human cDNA Sequence Project. 86J is the inventors' full-length cDNA, renamed as a 86F. 86S is a splicing variant of 86F identified by RT-PCR which is identical to 86F except for a 97 bp-deletion in the 3′-portion, corresponding to exon 7 of 86F, which is spliced out in 86S. 86Y, as illustrated in SEQ ID NOS: 3 and 6, is the full length 86F, but containing the extra 172 bp insertion in exon 9. FIGS. 1-3.

To better understand the function of the 86 polypeptide, the inventors performed a domain analysis using the SMART (Simple, Modular Architecture Research Tool) Program. The search results showed that there was a hidden ICE-p20 domain (residues 79-87) and three visible SEG domains. The ICE-p20 domain was reported to be a catalytic site of the caspase enzyme family, which is involved in apoptosis. The SEG domain implied a low complexity composition of amino acid sequence, and its function remains unknown (SEG 1=residues 3-21; SEG 2=37-65; SEG 3=375-388 (86F); 328-345 (86S)).

To examine expression of this gene in normal and tumor cells, the inventors performed RT-PCR. The inventors detected only full-length transcripts in normal cells, but a shortened form was observed in cancer cell lines and gliomas. Cloning and sequencing of the shorter form showed that it resulted from an alternatively spliced transcript (i.e., lacking exon 7).

In addition, two peptides were designed to generate antibodies against the 86 proteins. One, designated W86.46, comprised residues 48-62, and the other, designated W86.116, comprised residues 116-130. Two antibodies produced using these peptides identified highly specific bands with the sizes of 45 kD, 43 kD, and 36 kD, corresponding to variants 86F, 86S and 86Y, as expected. In a western blot format, using the cell line U251, the non-specific crossreactivity was not significant, with only a few bands observed around 65 kD.

### REFERENCES

The following references, to the extent that they provide exemplary procedural or other details supplementary to those set forth herein, are specifically incorporated herein by reference.

U.S. Pat. No. 4,196,265

U.S. Pat. No. 4,554,101

U.S. Pat. No. 4,683,195

U.S. Pat. No. 4,683,202

U.S. Pat. No. 4,683,202

U.S. Pat. No. 4,684,611

U.S. Pat. No. 4,800,159

U.S. Pat. No. 4,873,191

U.S. Pat. No. 4,879,236

U.S. Pat. No. 4,883,750

U.S. Pat. No. 4,952,500

U.S. Pat. No. 5,217,879

U.S. Pat. No. 5,279,721

U.S. Pat. No. 5,302,523

U.S. Pat. No. 5,322,783

U.S. Pat. No. 5,354,855

U.S. Pat. No. 5,384,253

U.S. Pat. No. 5,384,253

U.S. Pat. No. 5,464,765

U.S. Pat. No. 5,506,138

U.S. Pat. No. 5,538,877

U.S. Pat. No. 5,538,880

U.S. Pat. No. 5,550,318

U.S. Pat. No. 5,563,055

U.S. Pat. No. 5,580,859

U.S. Pat. No. 5,589,466

U.S. Pat. No. 5,610,042

U.S. Pat. No. 5,610,042

U.S. Pat. No. 5,656,610

U.S. Pat. No. 5,670,488

U.S. Pat. No. 5,702,932

U.S. Pat. No. 5,736,524

U.S. Pat. No. 5,739,018

U.S. Pat. No. 5,780,448

U.S. Pat. No. 5,789,215

U.S. Pat. No. 5,824,544

U.S. Pat. No. 5,830,725

U.S. Pat. No. 5,849,304

U.S. Pat. No. 5,851,826

U.S. Pat. No. 5,858,744

U.S. Pat. No. 5,871,982

U.S. Pat. No. 5,871,983

U.S. Pat. No. 5,871,986

U.S. Pat. No. 5,879,934

U.S. Pat. No. 5,888,502

U.S. Pat. No. 5,925,565

U.S. Pat. No. 5,928,906

U.S. Pat. No. 5,932,210

U.S. Pat. No. 5,935,819

U.S. Pat. No. 5,945,100

U.S. Pat. No. 5,955,331

U.S. Pat. No. 5,981,274

U.S. Pat. No. 5,994,136

U.S. Pat. No. 5,994,624

U.S. Pat. No. 6,013,516

Almendro, Bellon, Rius, Lastres, Langa, Corbi, Bernabeu, “Cloning of the human platelet endothelial cell adhesion molecule-1 promoter and its tissue-specific expression. Structural and functional characterization,” J. Immunol., 157(12):5411-5421, 1996.

Amado and Chen, “Lentiviral vectors—the promise of gene therapy within reach?” Science, 285(5428):674-6, 1999.

Amado, Mitsuyasu, Zack, “Gene therapy for the treatment of AIDS: animal models and human clinical experience,” Front Biosci, 4:D468-75, 1999.

Angel, Bauman, Stein, Dellus, Rahmsdorf, and Herrlich, “12-0-tetradecanoyl-phorbol-13-acetate induction of the human collagenase gene is mediated by an inducible enhancer element located in the 5′ flanking region,” Mol. Cell. Biol., 7:2256, 1987.

Angel, Imagawa, Chiu, Stein, Imbra, Rahmsdorf, Jonat, Herrlich, and Karin, “Phorbol ester-inducible genes contain a common cis element recognized by a TPA-modulated trans-acting factor,” Cell, 49:729, 1987

Atchison and Perry, “Tandem κ immunoglobulin promoters are equally active in the presence of the κ enhancer: Implications for model of enhancer function,” Cell, 46:253, 1986.

Atchison and Perry, “The role of the κ enhancer and its binding factor nf-κ β in the developmental regulation of κ gene transcription,” Cell, 48:121, 1987.

Ausubel, Brent, Kingston, Moore, Seidman, Smith, Struhl, In: Current Protocols in Molecular Biology, John, Wiley & Sons, Inc, New York, 1994.

Baneriji, Olson, Schaffner, “A lymphocyte-specific cellular enhancer is located downstream of the joining region in immunoglobulin heavy chain genes,” Cell, 33(3):729-740, 1983.

Baneriji, Rusconi, Schaffner, “Expression of a β-globin gene is enhanced by remote SV40 DNA sequences,” Cell, 27(2 Pt 1):299-308, 1981.

Barany and Merrifield, “The Peptides,” Gross and Meienhofer (eds.), Academic Press, New York, 1-284, 1979.

Batra, Guttridge, Brenner, Dubinett, Baldwin, Boucher, “IkappaBalpha gene transfer is cytotoxic to squamous-cell lung cancer cells and sensitizes them to tumor necrosis factor-alpha-mediated cell death,” Am J Respir Cell Mol Biol, 21(2):238-245, 1999.

Bello, Leone, Vaquero, de Campos, Kusak, Sarasa, Pestana, Rey, “Allelic loss at 1p and 19q frequently occurs in association and may represent early oncogenic events in oligodendroglial tumors,” Int J Cancer, 64(3):207-210, 1995.

Berkhout, Silverman, Jeang, “Tat trans-activates the human immunodeficiency virus through a nascent RNA target,” Cell, 59:273-282, 1989.

Bett, Prevec, Graham, “Packaging capacity and stability of human adenovirus type 5 vectors,” J Virol, 67(10):5911-21, 1993.

Bieche, Khodja, Lidereau, “Deletion mapping in breast tumor cell lines points to two distinct tumor-suppressor genes in the 1-32-pter region, of feleted regions (1p36.2) being located within the consensus region of LOH in neuroblastoma,” Oncol Rep., 5(1):267-272, 1998.

Bieche, Champeme, Lidereau, “A tumor suppressor gene on chromosome 1p32-pter controls the amplification of MYC family genes in breast cancer,” Cancer Res, 54(16):4274-4276, 1994.

Bilbao, Feng, Rancourt, Jackson Jr, Curiel, “Adenoviral/retroviral vector chimeras: a novel strategy to achieve high-efficiency stable transduction in vivo,” FASEB J, 11(8):624-634, 1997.

Blackwell, Miller, Douglas, Li, Reynolds, Carroll, Peters, Strong, Curiel, “Retargeting to EGFR enhances adenovirus infection efficiency of squamous cell carcinoma,” Arch Otolaryngol Head Neck Surg, 125(8):856-863, 1999.

Blanar, Baldwin, Flavell, and Sharp, “A gamma-interferon-induced factor that binds the interferon response sequence of the MHC class I gene, H-2Kb,” EMBO J., 8:1139, 1989.

Blomer, Naldini, Kafri, Trono, Verma, Gage, “Highly efficient and sustained gene transfer in adult neurons with a lentivirus vector,” J. Virol., 71(9):6641-6649, 1997.

Bodine and Ley, “An enhancer element lies 3′ to the human a gamma globin gene,” EMBO J., 6:2997, 1987.

Bomme, Bardi, Pandis, Fenger, Kronborg, Heim, “Clonal karyotypic abnormalities in colorectal adenomas: clues to the early genetic events in the adenoma-carcinoma sequence,” Genes Chromosomes Cancer, 10(3):190-196, 1994.

Borg, Zhang, Olsson, Wenngren, “Chromosome 1 alterations in breast cancer: allelic loss on 1p and 1q is related to lymphogenic metastases and poor prognosis,” Genes Chromosomes Cancer, 5(4):311-320, 1992.

Boshart, Weber, Jahn, Dorsch-Hasler, Fleckenstein, and Schaffner, “A very strong enhancer is located upstream of an immediate early gene of human cytomegalovirus,” Cell, 41:521, 1985.

Bosze, Thiesen, Charnay, “A transcriptional enhancer with specificity for erythroid cells is located in the long terminal repeat of the Friend murine leukemia virus,” EMBO J., 5(7):1615-1623, 1986.

Braddock, Chambers, Wilson, Esnouf, Adams, Kingsman, and Kingsman, “HIV-I Tat activates presynthesized RNA in the nucleus,” Cell, 58:269, 1989.

Bulla and Siddiqui, “The hepatitis B virus enhancer modulates transcription of the hepatitis B virus surface-antigen gene from an internal location,” J. Virol., 62:1437, 1986.

Campbell and Villarreal, “Functional analysis of the individual enhancer core sequences of polyoma virus: cell-specific uncoupling of DNA replication from transcription,” Mol. Cell. Biol., 8:1993, 1988.

Campbell, In: Monoclonal Antibody Technology, Laboratory Techniques in Biochemistry and Molecular Biology, Vol. 13, Burden and Von Knippenberg (Eds.), Elseview, Amsterdam, pp. 71-74; 75-83, 1984.

Campere and Tilghman, “Postnatal repression of the α-fetoprotein gene is enhancer independent,” Genes and Dev., 3:537, 1989.

Campo, Spandidos, Lang, Wilkie, “Transcriptional control signals in the genome of bovine papilloma virus type 1,” Nature, 303:77, 1983.

Capaldi, Bell, Branchek, “Changes in order of migration of polypeptides in complex III and cytochrome C oxidase under different conditions of SDS polyacrylamide gel electrophoresis,” Biochem. Biophys. Res. Comm., 74(2):425-433, 1977.

Caplen, Higginbotham, Scheel, Vahanian, Yoshida, Hamada, Blaese, Ramsey, “Adeno-retroviral chimeric viruses as in vivo transducing agents,” Gene Ther, 6(3):454-459, 1999.

Carbonelli, Corley, Seigelchifer, Zorzopulos, “A plasmid vector for isolation of strong promoters in Escherichia coli,” FEMS Microbiol Lett, 177(1):75-82, 1999.

Case, Price, Jordan, Yu, Wang, Bauer, Haas, Xu, Stripecke, Naldini, Kohn, Crooks, “Stable transduction of quiescent CD34(+)CD38(−) human hematopoietic cells by HIV-1-based lentiviral vectors,” Proc Natl Acad Sci USA, 96(6):2988-2993, 1999.

Celander and Haseltine, “Glucocorticoid regulation of murine leukemia virus transcription elements is specified by determinants within the viral enhancer region,” J. Virology, 61:269, 1987.

Celander, Hsu, and Haseltine, “Regulatory Elements Within the Murine Leukemia Virus Enhancer Regions Mediate Glucocorticoid Responsiveness,” J. Virology, 62:1314, 1988.

Chandler, Maler, and Yamamoto, “DNA sequences bound specifically by glucocorticoid receptor in vitro render a heterlogous promoter hormone responsive in vivo,” Cell, 33:489, 1983.

Chandler, Mayeda, Yeakley, Krainer, Fu, “RNA splicing specificity determined by the coordinated action of RNA recognition motifs in SR proteins,” Proc Natl Acad Sci USA, 94(8):3596-601, 1997.

Chang, Erwin, and Lee, “Glucose-regulated Protein (GRP94 and GRP78) Genes Share Common Regulatory Domains and are Coordinately Regulated by Common Trans-acting Factors,” Mol. Cell. Biol., 9:2153, 1989.

Chatterjee, Lee, Rentoumis, and Jameson, “Negative regulation of the thyroid-stimulating hormone alpha gene by thyroid hormone: receptor interaction adjacent to the TATA box,” Proc Natl. Acad Sci. U.S.A., 86:9114, 1989.

Chen and Okayama, “High-efficiency transformation of mammalian cells by plasmid DNA,” Mol. Cell Biol., 7(8):2745-2752, 1987.

Chillon, Bosch, Zabner, Law, Armentano, Welsh, Davidson, “Group D adenoviruses infect primary central nervous system cells more efficiently than those from group C,” J Virol, 73(3):2537-2540, 1999.

Choi, Chen, Kriegler, and Roninson, “An altered pattern of cross-resistance in multi-drug-resistant human cells results from spontaneous mutations in the mdr-1 (p-glycoprotein) gene,” Cell, 53:519, 1988.

Cocea, “Duplication of a region in the multiple cloning site of a plasmid vector to enhance cloning-mediated addition of restriction sites to a DNA fragment,” Biotechniques, 23(5):814-816, 1997.

Coffey, Strong, Forsyth, Lee, “Reovirus therapy of tumors with activated Ras pathway,” Science, 282(5392):1332-1334, 1998.

Cohen, Walter, and Levinson, “A Repetitive Sequence Element 3′ of the Human c-Ha-ras1 Gene Has Enhancer Activity,” J. Cell. Physiol., 5:75, 1987.

Costa, Lai, Grayson, and Darnell, “The cell-specific enhancer of the mouse transthyretin (prealbumin) gene binds a common factor at one site and a liver-specific factor(s) at two other sites,” Mol. Cell. Biol., 8:81-90, 1988.

Cripe, Haugen, Turk, Tabatabai, Schmid, Durst, Gissmann, Roman, and Turek, “Transcriptional Regulation of the Human Papilloma Virus-16 E6-E7 Promoter by a Keratinocyte-Dependent Enhancer, and by Viral E2 Trans-Activator and Repressor Gene Products: Implications for Cervical Carcinogenesis,” EMBO J., 6:3745, 1987.

Culotta and Hamer, “Fine Mapping of a Mouse Metallothionein Gene Metal-Response Element,” Mol. Cell. Biol., 9:1376-1380, 1989.

Culver, Ram, Wallbridge, Ishii, Oldfield, Blaese, “In vivo gene transfer with retroviral vector-producer cells for treatment of experimental brain tumors,” Science, 256(5063):1550-1552, 1992.

Dandolo, Blangy, and Kamen, “Regulation of Polyma Virus Transcription in Murine Embryonal Carcinoma Cells,” J. Virology, 47:55-64, 1983.

De Villiers, Schaffner, Tyndall, Lupton, and Kamen, “Polyoma Virus DNA Replication Requires an Enhancer,” Nature, 312(5991):242-246, 1984.

DeLuca, McCarthy, Schaffer. “Isolation and characterization of deletion mutants of herpes simplex virus type 1 in the gene encoding immediate-early regulatory protein ICP4,” J Virol, 56(2):558-570, 1985.

Derby, Sena-Esteves, Breakefield, Corey, “Gene transfer into the mammalian inner ear using HSV-1 and vaccinia virus vectors,” Hear Res, 134(1-2):1-8, 1999.

Deschamps, Meijlink, and Verma, “Identification of a transcriptional enhancer element upstream from the proto-oncogene fos,” Science, 230:1174-1177, 1985.

Edbrooke, Burt, Cheshire, and Woo, “Identification of cis-acting sequences responsible for phorbol ester induction of human serum amyloid a gene expression via a nuclear-factor-kappa β-like transcription factor,” Mol. Cell. Biol., 9:1908-1916, 1989.

Edlund, Walker, Barr, and Rutter, “Cell-specific expression of the rat insulin gene: evidence for role of two distinct 5′ flanking elements,” Science, 230:912-916, 1985.

Engel and Kohn, “Stem cell directed gene therapy,” Front Biosci, 4:e26-33, 1999.

European Patent No. 0 273,085

European Patent No. 329,822

Fechheimer, Boylan, Parker, Sisken, Patel, Zimmer, “Transfection of mammalian cells with plasmid DNA by scrape loading and sonication loading,” Proc. Nat'l Acad. Sci. USA, 84:8463-8467, 1987.

Feldman, Tahlil, Steg, “Adenovirus-mediated arterial gene therapy for restenosis problems and perspectives,” Semin. Interv. Cardiol., 1(3):203-208, 1997.

Feng and Holland, “HIV-I Tat Trans-Activation Requires the Loop Sequence Within Tar,” Nature, 334:6178, 1988.

Feng, Jackson Jr, Goldman, Rancourt, Wang, Dusing, Siegal, Curiel, “Stable in vivo gene transduction via a novel adenoviral/retroviral chimeric vector,” Nat Biotechnol, 15(9):866-70, 1997.

Firak and Subramanian, “Minimal Transcription Enhancer of Simian Virus 40 is a 74-Base-Pair Sequence that Has Interacting Domains,” Mol Cell. Biol., 6:3667, 1986.

Fodor, Rava, Huang, Pease, Holmes, Adams, “Multiplexed biochemical assays with biological chips,” Nature, 364:555-556, 1993..

Foecking and Hofstetter, “Powerful and Versatile Enhancer-Promoter Unit for Mammalian Expression Vectors,” Gene, 45(1):101-105, 1986.

Forster and Symons, “:Self-cleavage of plus and minus RNAs of a virusoid and a structural model for the active sites,” Cell, 49(2):211-220, 1987.

Fraley, Fomari, Kaplan, “Entrapment of a bacterial plasmid in phospholipid vesicles: potential for gene transfer,” Proc. Nat'l Acad. Sci. USA, 76:3348-3352, 1979.

Freifelder, Physical Biochemistry Applications to Biochemistry and Molecular Biology, 2nd ed. Wm. Freeman and Co., New York, N.Y., 1982.

Frohman, In: PCR Protocols: A Guide To Methods And Applications, Academic Press, N.Y., 1990.

Fujita, Shibuya, Hotta, Yamanishi, and Taniguchi, “Interferon-Beta Gene Regulation: Tandemly Repeated Sequences of a Synthetic 6-bp Oligomer Function as a Virus-Inducible Enhancer,” Cell, 49:357, 1987.

Fujiwara and Tanaka, “Molecular surgery for human colorectal cancer with tumor suppressor p53 gene transfer,” Nippon Geka Gakkai Zasshi, 99(7):463-468, 1998.

Fuller, Rhee, Hess, Caskey, Wang, Bruner, Yung, Zhang, “Reactivation of insulin-like growth factor binding protein 2 expression in glioblastoma multiforme: a revelation by parallel gene expression profiling,” Cancer Res, 59(17):4228-32, 1999.

Garoff H, Li, “Recent advances in gene expression using alphavirus vectors,” Curr Opin Biotechnol, 9(5):464-469, 1998.

Garrido, Carnicero, Lim, Schimmang, “Differential effects on the survival of neuronal and non-neuronal cells after infection by herpes simplex virus type 1 mutants,” J. Neurovirol., 5(3):280-288, 1999.

GB Application No. 2,202,328

Gefter, Margulies, Scharff, “A simple method for polyethylene glycol-promoted hybridization of mouse myeloma cells,” Somatic Cell Genet, 3(2):231-6, 1977.

Gerlach et al., Nature (London), 328:802-805, 1987.

Ghosh and Bachhawat, Targeting of Liposomes to Hepatocytes. In: Liver Diseases, Targeted Diagnosis and Therapy Using Specific Receptors and Ligands. Wu et al., eds., Marcel Dekker, New York, pp. 87-104, 1991.

Gilles, Morris, Oi, and Tonegawa, “A tissue-specific transcription enhancer element is located in the major intron of a rearranged immunoglobulin heavy-chain gene,” Cell, 33:717, 1983.

Gloss, Bernard, Seedorf, and Klock, “The upstream regulatory region of the human papilloma virus-16 contains an E2 protein-independent enhancer which is specific for cervical carcinoma cells and regulated by glucocorticoid hormones,” EMBO J., 6:3735, 1987.

Gnant, Noll, Irvine, Puhlmann, Terrill, Alexander Jr, Bartlett, “Tumor-specific gene delivery using recombinant vaccinia virus in a rabbit model of liver metastases,” J Natl Cancer Inst, 91(20):1744-1750, 1999.

Gnant, Puhlmann, Bartlett, Alexander Jr., “Regional versus systemic delivery of recombinant vaccinia virus as suicide gene therapy for murine liver metastases,” Ann Surg, 230(3):352-360, 1999.

Godbout, Ingram, and Tilghman, “Fine-Structure Mapping of the Three Mouse Alpha-Fetoprotein Gene Enhancers,” Mol. Cell. Biol., 8:1169, 1988.

Goding, In: Monoclonal Antibodies: Principles and Practice, 2d ed., Academic Press, Orlando, Fla., pp. 60-61, and 71-74, 1986.

Goodboum and Maniatis, “Overlapping Positive and Negative Regulatory Domains of the Human β-Interferon Gene,” Proc. Natl. Acad. Sci. USA, 85:1447, 1988.

Goodbourn, Burstein, and Maniatis, “The Human Beta-Interferon Gene Enhancer is Under Negative Control,” Cell, 45:601, 1986.

Gopal, “Gene transfer method for transient gene expression, stable transformation, and cotransformation of suspension cell cultures,” Mol. Cell Biol., 5:1188-1190, 1985.

Graham and Prevec, “Methods for construction of adenovirus vectors,” Mol Biotechnol, 3(3):207-220, 1995.

Graham and van der Eb, “A new technique for the assay of infectivity of human adenovirus 5 DNA,” Virology, 52:456-467, 1973.

Greene, Bohnlein, and Ballard, “HIV-1, and Normal T-Cell Growth: Transcriptional Strategies and Surprises,” Immunology Today, 10:272, 1989

Greene, Bohnlein, and Ballard, “HIV-1, and Normal T-Cell Growth: Transcriptional Strategies and Surprises,” Immunology Today, 10:272, 1989

Grosschedl and Baltimore, “Cell-type specificity of immunoglobulin gene expression is regulated by at least three dna sequence elements,” Cell, 41:885, 1985.

Hacia, Brody, Chee, Fodor, Collins, “Detection of heterozygous mutations in BRCA1 using high density oligonucleotide arrays and two-colour fluorescence analysis,” Nature Genet., 14:441-449, 1996.

Haecker, Stedman, Balice-Gordon, Smith, Greelish, Mitchell, Wells, Sweeney, Wilson, “In vivo expression of full-length human dystrophin from adenoviral vectors deleted of all viral genes,” Hum Gene Ther, 7(15):1907-14, 1996.

Han, Semba, Abe, Makino, Furukawa, Fukushige, Takahashi, Sakurada, Sato, Shiiba, Matsuno, Nimura, Nakagawara, Horii, “Infrequent somatic mutations of the p73 gene in various human cancers,” Eur J Surg Oncol, 25(2):194-198, 1999.

Harland and Weintraub, “Translation of mRNA injected into Xenopus oocytes is specifically inhibited by antisense RNA,” J. Cell Biol., 101:1094-1099, 1985.

Haslinger and Karin, “Upstream Promoter Element of the Human Metallothionein-II Gene Can Act Like an Enhancer Element,” Proc Natl. Acad. Sci. U.S.A., 82:8572, 1985.

Hauber and Cullen, “Mutational Analysis of the Trans-Activiation-Responsive Region of the Human Immunodeficiency Virus Type I Long Terminal Repeat,” J. Virology, 62:673, 1988.

Hen, Borrelli, Fromental, Sassone-Corsi, and Chambon, “A Mutated Polyoma Virus Enhancer Which is Active in Undifferentiated Embryonal Carcinoma Cells is not Repressed by Adenovirus-2 E1A Products,” Nature, 321:249, 1986.

Hensel, Meichle, Pfizenmaier, and Kronke, “PMA-responsive 5′ flanking sequences of the human TNF gene,” Lymphokine Res., 8:347, 1989.

Hermens and Verhaagen, “Viral vectors, tools for gene transfer in the nervous system,” Prog Neurobiol, 55(4):399-432, 1998.

Herr and Clarke, “The SV40 Enhancer is Composed of Multiple Functional Elements That Can Compensate for One Another,” Cell, 45:461, 1986.

Hirochika, Browker, and Chow, “Enhancers and Trans-Acting E2 Transcriptional Factors of Papilloma Viruses,” J. Virol., 61:2599, 1987.

Hirsch, Gaugler, Deagostini-Bauzin, Bally-Cuif, and Gordis, “Identification of positive and negative regulatory elements governing cell-type-specific expression of the neural-cell-adhesion-molecule gene,” Mol. Cell. Biol., 10:1959, 1990.

Ho and Baxter, “Insulin-like growth factor-binding protein-2 in patients with prostate carcinoma and benign prostatic hyperplasia,” Clin Endocrinol, 46(3):333-342, 1997.

Holbrook, Gulino, and Ruscetti, “cis-Acting Transcriptional Regulatory Sequences in the Gibbon Ape Leukemia Virus (GALV) Long Terminal Repeat,” Virology, 157:211, 1987.

Holzer, Mayrhofer, Gritschenberger, Domer, Falkner, “Poxviral/retroviral chimeric vectors allow cytoplasmic production of transducing defective retroviral particles,” Virology, 253(1):107-114, 1999.

Horlick and Benfield, “The Upstream Muscle-Specific Enhancer of the Rat Muscle Creatine Kinase Gene is Composed of Multiple Elements,” Mol. Cell. Biol., 9:2396, 1989.

Howard, Kalthoff, Fong, “Ablation of tumor cells in vivo by direct injection of HSV-thymidine kinase retroviral vector and ganciclovir therapy,” Ann. NY Acad. Sci., 880:352-365, 1999.

Huang, Ostrowski, Berard, and Hagar, “Glucocorticoid regulation of the ha-musv p21 gene conferred by sequences from mouse mammary tumor virus,” Cell, 27:245, 1981.

Huard, Krisky, Oligino, Marconi, Day, Watkins, Glorioso, “Gene transfer to muscle using herpes simplex virus-based vectors,” Neuromuscul Disord, 7(5):299-313, 1997.

Hug, Costas, Staeheli, Aebi, and Weissmann, “Organization of the Murine Mx Gene and Characterization of its Interferon- and Virus-Inducible Promoter,” Mol. Cell. Biol., 8:3065, 1988.

Hwa and Rosenfeld, “The insulin-like growth factor-binding protein (IGFBP) superfamily,” Endocr Rev, 20(6):761-787, 1999.

Hwang, Lim, and Chae, “Characterization of the S-Phase-Specific Transcription Regulatory Elements in a DNA-Replication-Independent Testis-Specific H2B (TH2B) Histone Gene,” Mol. Cell. Biol., 10:585, 1990.

Ichimiya, Nimura, Kageyama, Takada, Sunahara, Shishikura, Nakamura, Sakiyama, Seki, Ohira, Kaneko, McKeon, Caput, Nakagawara, “p73 at chromosome 1p36.3 is lost in advanced stage neuroblastoma but its mutation is infrequent,” Oncogene, 18(4):1061-1066, 1999.

Imagawa, Chiu, and Karin, “Transcription Factor AP-2 Mediates Induction by Two Different Signal-Transduction Pathways: Protein Kinase C and cAMP,” Cell, 51:251, 1987.

Imai, Akagi, Isaka, “Towards gene therapy for renal diseases,” Nephrologie, 19(7):397-402, 1998.

Imbra and Karin, “Phorbol Ester Induces the Transcriptional Stimulatory Activity of the SV40 Enhancer,” Nature, 323:555, 1986.

Imler, Lemaire, Wasvlyk, and Waslyk, “Negative Regulation Contributes to Tissue Specificity of the Immunoglobulin Heavy-Chain Enhancer,” Mol. Cell. Biol, 7:2558, 1987.

Imperiale and Nevins, “Adenovirus 5 E2 Transcription Unit: an E1A-Inducible Promoter with an Essential Element that Functions Independently of Position or Orientation,” Mol. Cell. Biol., 4:875, 1984.

Innis et al., PCR Protocols, Academic Press, Inc., San Diego Calif., 1990.

Ino, Zlatescu, Sasaki, Macdonald, Stemmer-Rachamimov, Jhung, Ramsay, von Deimling, Louis, Cairncross, “Long survival and therapeutic responses in patients with histologically disparate high-grade gliomas demonstrating chromosome 1p loss,” J Neurosurg, 92(6):983-990, 2000.

Irie, Anderegg, Kashani-Sabet, Ohkawa, Suzuki, Halks-Miller, Curiel, Scanlon, “Therapeutic efficacy of an adenovirus-mediated anti-H-ras ribozyme in experimental bladder cancer,” Antisense Nucleic Acid Drug Dev, 9(4):341-349, 1999.

Jakobovits, Smith, Jakobovits, and Capon, “A Discrete Element 3′ of Human Immunodeficiency Virus 1 (HIV-1) and HIV-2 mRNA Initiation Sites Mediates Transcriptional Activation by an HIV Trans-Activator,” Mol. Cell. Biol., 8:2555, 1988.

Jameel and Siddiqui, “The Human Hepatitis B Virus Enhancer Requires Transacting Cellular Factor(s) for Activity,” Mol. Cell. Biol., 6:710, 1986.

Jaynes, Johnson, Buskin, Gartside, and Hauschka, “The Muscle Creatine Kinase Gene is Regulated by Multiple Upstream Elements, Including a Muscle-Specific Enhancer,” Mol. Cell. Biol., 8:62, 1988.

Johnson et al., Peptide Turn Mimetics” IN: Biotechnology And Pharmacy, Pezzuto et al, eds., Chapman and Hall, New York, 1993.

Johnson, Wold, and Hauschka, “Muscle creatine kinase sequence elements regulating skeletal and cardiac muscle expression in transgenic mice,” Mol. Cell. Biol., 9:3393, 1989.

Johnston, Gasmi, Lim, Elder, Yee, Jolly, Campbell, Davidson, Sauter, “Minimum requirements for efficient transduction of dividing and nondividing cells by feline immunodeficiency virus vectors,” J Virol, 73(6):4991-5000, 1999.

Joyce, “RNA evolution and the origins of life,” Nature, 338:217-244, 1989.

Kadesch and Berg, “Effects of the Position of the Simian Virus 40 Enhancer on Expression of Multiple Transcription Units in a Single Plasmid,” Mol. Cell. Biol., 6:2593, 1986.

Kaneda, Iwai, Uchida, “Increased expression of DNA cointroduced with nuclear protein in adult rat liver,” Science, 243:375-378, 1989.

Kanety, Kattan, Goldberg, Kopolovic, Ravia, Menczer, Karasik, “Increased insulin-like growth factor binding protein-2 (IGFBP-2) gene expression and protein production lead to high IGFBP-2 content in malignant ovarian cyst fluid,” Br J Cancer, 73(9): 1069-73, 1996.

Kanety, Madjar, Dagan, Levi, Papa, Pariente, Goldwasser, Karasik, “Serum insulin-like growth factor-binding protein-2 (IGFBP-2) is increased and IGFBP-3 is decreased in patients with prostate cancer: correlation with serum prostate-specific antigen,” J Clin Endocrinol Metab, 77(l):229-233, 1993.

Karasik, Menczer, Pariente, Kanety, “Insulin-like growth factor-I (IGF-I) and IGF-binding protein-2 are increased in cyst fluids of epithelial ovarian cancer,” J Clin Endocrinol Metab, 78(2):271-276, 1994.

Karin, Haslinger, Heguy, Dietlin, and Cooke, “Metal-Responsive Elements Act as Positive Modulators of Human Metallothionein-IIA Enhancer Activity,” Mol. Cell. Biol., 7:606, 1987.

Katinka, Vasseur, Montreau, Yaniv, and Blangy, “Polyoma DNA Sequences Involved in the Control of Viral Gene Expression in Murine Embryonal Carcinoma Cells,” Nature, 290:720, 1981.

Katinka, Vasseur, Montreau, Yaniv, and Blangy, “Polyoma DNA Sequences Involved in the Control of Viral Gene Expression in Murine Embryonal Carcinoma Cells,” Nature, 290:720, 1981.

Katinka, Yaniv, Vasseur, and Blangy, “Expression of Polyoma Early Functions in Mouse Embryonal Carcinoma Cells Depends on Sequence Rearrangements in the Beginning of the Late Region,” Cell, 20:393, 1980.

Kato et al., J. Biol. Chem., 266:3361-3364, 1991.

Kaufman, Jia, Tan, Chen, Gabelt, Booth, Tufaro, Cynader, “A perspective of gene therapy in the glaucomas,” Surv. Ophthalmol., 43(l):S91-97, 1999.

Kawamoto, Makino, Niw, Sugiyama, Kimura, Anemura, Nakata, and Kakunaga, “Identification of the Human Beta-Actin Enhancer and its Binding Factor,” Mol. Cell. Biol., 8:267, 1988.

Kay, “Hepatic gene therapy for haemophilia B,” Haemophilia, 4(4):389-392, 1998.

Kiledjian, Su, Kadesch, “Identification and characterization of two functional domains within the murine heavy-chain enhancer,” Mol. Cell. Biol., 8:145, 1988.

Kim and Chech, “Three-dimensional model of the active site of the self-splicing rRNA precursor of Tetrahymena,” Proc. Natl. Acad. Sci. USA, 84(24):8788-8792, 1987.

Klamut, Gangopadyhay, Worton, and Ray, “Molecular and Functional Analysis of the Muscle-Specific Promoter Region of the Duchenne Muscular Dystrophy Gene,” Mol. Cell. Biol., 10:193, 1990.

Klein, Gu, Goddard, Rosenthal, “Selection for genes encoding secreted proteins and receptors,” Proc. Natl. Acad. Sci., 93:7108-7113, 1996.

Klimatcheva, Rosenblatt, Planelles, “Lentiviral vectors and gene therapy,” Front Biosci, 4:D481-496, 1999.

Kohut, Davis, Jackson, Jani, Ghaffar, Mayer, Essig, “Exercise effects on IFN-beta expression and viral replication in lung macrophages after HSV-1 infection,” Am. J. Physiol., 275(6Ptl):L1089-1094, 1998.

Kooby, Carew, Halterman, Mack, Bertino, Blumgart, Federoff, Fong, “Oncolytic viral therapy for human colorectal cancer and liver metastases using a multi-mutated herpes simplex virus type-1 (G207), FASEB J., 13(11):1325-1334, 1999.

Koch, Benoist, and Mathis, “Anatomy of a new β-cell-specific enhancer,” Mol. Cell. Biol., 9:303, 1989.

Kohler and Milstein, “Continuous cultures of fused cells secreting antibody of predefined specificity,” Nature, 256(5517):495-7, 1975.

Kohler and Milstein, “Derivation of specific antibody-producing tissue culture and tumor lines by cell fusion,” Eur J Immunol, 6(7):511-9, 1976.

Kovacs, Soudah, Hoene, “Binucleated cells in a human renal cell carcinoma with 34 chromosomes,” Cancer Genet Cytogenet, 31(2):211-215, 1988.

Kraus, Woltje, Schonwetter, Hollt, “Alternative promoter usage and tissue specific expression of the mouse somatostatin receptor 2 gene,” FEBS Lett, 428(3):165-170, 1998.

Kriegler and Botchan, “A retrovirus LTR contains a new type of eukaryotic regulatory element,” In: Eukaryotic Viral Vectors, Gluzman (ed.), Cold Spring Harbor, Cold Spring Harbor Laboratory, NY, 1982.

Kriegler and Botchan, “Enhanced transformation by a simian virus 40 recombinant virus containing a Harvey murine sarcoma virus long terminal repeat,” Mol. Cell. Biol. 3:325, 1983.

Kriegler et al., “Promoter substitution and enhancer augmentation increases the penetrance of the sv40 a gene to levels comparable to that of the harvey murine sarcoma virus ras gene in morphologic transformation,” In: Gene Expression, Alan Liss (Ed.), Hamer and Rosenberg, N.Y., 1983.

Kriegler et al., “Viral Integration and Early Gene Expression Both Affect the Efficiency of SV40 Transformation of Murine Cells: Biochemical and Biological Characterization of an SV40 Retrovirus,” In: Cancer Cells 2/Oncogenes and Viral Genes, Van de Woude et al. eds, Cold Spring Harbor: Cold Spring Harbor Laboratory, 1984.

Kriegler, Perez, Hardy and Botchan, “Transformation mediated by the sv40 t antigens: separation of the overlapping sv40 early genes with a retroviral vector,” Cell, 38:483, 1984.

Krisky, Marconi, Oligino, Rouse, Fink, Cohen, Watkins, Glorioso, “Development of herpes simplex virus replication-defective multigene vectors for combination gene therapy applications,” Gene Ther, 5(11):1517-1530, 1998.

Krisky, Wolfe, Goins, Marconi, Ramakrishnan, Mata, Rouse, Fink, Glorioso, “Deletion of multiple immediate-early genes from herpes simplex virus reduces cytotoxicity and permits long-term gene expression in neurons,” Gene Ther, 5(12):1593-1603, 1998.

Kuhl, De La Fuenta, Chaturvedi, Parinool, Ryals, Meyer, and Weissman, “Reversible Silencing of Enhancers by Sequences Derived From the Human IFN-alpha Promoter,” Cell, 50:1057, 1987.

Kunz, Zimmerman, Heisig, and Heinrich, “Identification of the Promoter Sequences Involved in the Interleukin-6-Dependent Expression of the Rat Alpha-2-Macroglobulin Gene,” Nucl. Acids Res., 17:1121, 1989.

Kwoh, Davis, Whitfield, Chappelle, DiMichele, Gingeras, “Transcription-based amplification system and detection of amplified human immunodeficiency virus type 1 with a bead-based sandwich hybridization format,” Proc. Nat. Acad. Sci. USA, 86: 1173, 1989

Kyte and Doolittle, “A simple method for displaying the hydropathic character of a protein,” J Mol Biol, 57(1):105-32, 1982.

Lachmann and Efstathiou, “Gene transfer with herpes simplex vectors, Curr. Opin. Mol. Ther., 1(5):622-632, 1999.

Lareyre, Thomas, Zheng, Kasper, Ong, Orgebin-Crist, Matusik, “A 5-kilobase pair promoter fragment of the murine epididymal retinoic acid-binding protein gene drives the tissue-specific, cell-specific, and androgen-regulated expression of a foreign gene in the epididymis of transgenic mice,” J Biol Chem, 274(12):8282-8290, 1999.

Larsen, Harney, and Moore, “Repression medaites cell-type-specific expression of the rat growth hormone gene,” Proc Natl. Acad. Sci. USA., 83:8283, 1986.

Laspia, Rice, and Mathews, “HIV-1 Tat protein increases transcriptional initiation and stabilizes elongation,” Cell, 59:283, 1989.

Latimer, Berger, and Baumann, “Highly conserved upstream regions of the alpha..sub.1-antitrypsin gene in two mouse species govern liver-specific expression by different mechanisms,” Mol. Cell. Biol., 10:760, 1990.

Lee, Bookstein, Hong, Young, Shew, Lee, “Human retinoblastoma susceptibility gene: cloning, identification, and sequence,” Science, 13:235(4794):1394-1399, 1987.

Lee, Mulligan, Berg, and Ringold, “Glucocorticoids Regulate Expression of Dihydrofolate Reductase cDNA in Mouse Mammary Tumor Virus Chimaeric Plasmids,” Nature, 294:228, 1981.

Lee, Wang, Yajima, Jose, Mouradian, “Tissue-specific promoter usage in the D1A dopamine receptor gene in brain and kidney,” DNA Cell Biol, 16(11):1267-75, 1997.

Leibowitz, Beattie, Kafri, Cirulli, Lopez, Hayek, Levine, “Gene transfer to human pancreatic endocrine cells using viral vectors,” Diabetes, 48(4):745-753, 1999.

Lesch, “Gene transfer to the brain: emerging therapeutic strategy in psychiatry?” Biol Psychiatry, 45(3):247-253, 1999.

Levenson, Transue, Roninson, “Internal ribosomal entry site-containing retroviral vectors with green fluorescent protein and drug resistance markers,” Hum Gene Ther, 9(8):1233-6, 1998.

Levinson, Khoury, VanDeWoude, and Gruss, “Activation of SV40 Genome by 72-Base-Pair Tandem Repeats of Moloney Sarcoma Virus,” Nature, 295:79, 1982.

Lin, Cross, Halden, Dragos, Toledano, and Leonard, “Delineation of an enhancerlike positive regulatory element in the interleukin-2 receptor .alpha.-chain gene,” Mol. Cell. Biol., 10:850, 1990.

Lo Cunsolo, Iolascon, Cavazzana, Cusano, Strigini, Mazzocco, Giordani, Massimo, De Bernardi, Conte, Tonini, “Neuroblastoma in two siblings supports the role of 1p36 deletion in tumor development,” Cancer Genet Cytogenet, 109(2):126-130, 1999.

Lundstrom, “Alphaviruses as tools in neurobiology and gene therapy,” J Recept Signal Transduct Res, 19(1-4):673-686, 1999.

Luria, Gross, Horowitz, and Givol, “Promoter Enhancer Elements in the Rearranged Alpha-Chain Gene of the Human T-Cell Receptor,” EMBO J., 6:3307, 1987.

Lusky and Botchan, “Transient Replication of Bovine Papilloma Virus Type 1 Plasmids: cis and trans Requirements,” Proc Natl. Acad. Sci. US.A., 83:3609, 1986.

Lusky, Berg, Weiher, and Botchan, “Bovine Papilloma Virus Contains an Activator of Gene Expression at the Distal End of the Early Transcription Unit,” Mol. Cell. Biol. 3:1108, 1983.

Macejak and Sarnow, “Internal initiation of translation mediated by the 5′ leader of a cellular mRNA,” Nature, 353:90-94, 1991.

Majors and Varmus, “A small region of the mouse mammary tumor virus long terminal repeat confers glucocorticoid hormone regulation on a linked heterologous gene,” Proc. Natl. Acad. Sci. USA, 80:5866, 1983.

Maniatis, et al., Molecular Cloning, A Laboratory Manual, Cold Spring Harbor Press, Cold Spring Harbor, N.Y., 1988.

Manipulating the Mouse Embryo, A Laboratory Manual, 2^{ND} Edition Hogan, Beedington, Costantimi and Long, (Eds), Cold Spring Harbor Laboratory Press, 1994

Manni, Badger, Wei, Zaenglein, Grove, Khin, Heitjan, Shimasaki, Ling, “Hormonal regulation of insulin-like growth factor II and insulin-like growth factor binding protein expression by breast cancer cells in vivo: evidence for stromal epithelial interactions,” Cancer Res, 54(11):2934-2942, 1994.

Marienfeld, Haack, Thalheimer, Schneider-Rasp, Brackmann Poller, “‘Autoreplication’ of the vector genome in recombinant adenoviral vectors with different E1 region deletions and transgenes,” Gene Ther, 6(6):1101-1113, 1999.

Martin and Baxter, “Oncogenic ras causes resistance to the growth inhibitor insulin-like growth factor binding protein-3 (IGFBP-3) in breast cancer cells,” J Biol Chem, 274(23):16407-16411, 1999.

Mastrangelo, Zou, Hardwick, Betenbaugh, “Antiapoptosis chemicals prolong productive lifetimes of mammalian cells upon Sindbis virus vector infection,” Biotechnol Bioeng, 65(3):298-305, 1999.

McNeall, Sanchez, Gray, Chesterman, and Sleigh, “Hyperinducible Gene Expression From a Metallotionein Promoter Containing Additional Metal-Responsive Elements,” Gene, 76:81, 1989.

Merrifield, “Solid phase synthesis,” Science, 232(4748):341-347, 1986.

Michel and Westhof, “Modelling of the three-dimensional architecture of group I catalytic introns based on comparative sequence analysis,” J. Mol. Biol., 216:585-610, 1990.

Miksicek, Heber, Schmid, Danesch, Posseckert, Beato, and Schutz, “Glucocorticoid Responsiveness of the Transcriptional Enhancer of Moloney Murine Sarcoma Virus,” Cell, 46:203, 1986.

Miller, Chumakov, Said, Chen, Aslo, Koeffler, “Mutant p53 proteins have diverse intracellular abilities to oligomerize and activate transcription,” Oncogene, 8(7):1815-1824, 1993.

Millikan, Ingles, Diep, Xue, Zhou, Florentine, Sparkes, Haile, “Linkage analysis and loss of heterozygosity for chromosome arm 1p in familial breast cancer,” Genes Chromosomes Cancer, 25(4):354-361, 1999.

Mishra, Bass, Ooi, Sidawy, Korman, “Role of insulin-like growth factor-I (IGF-I) receptor, IGF-I, and IGF binding protein-2 in human colorectal cancers,” Growth Horm IGF Res, 8(6):473-9, 1998.

Moldawer, Edwards, Josephs, Minter, Copeland, MacKay, “Application of gene therapy to acute inflammatory diseases,” Shock, 12(2):83-101, 1999.

Mordacq and Linzer, “Co-localization of elements required for phorbol ester stimulation and glucocorticoid repression of proliferin gene expression,” Genes and Dev., 3:760, 1989.

Mordacq and Linzer, “Co-localization of elements required for phorbol ester stimulation and glucocorticoid repression of proliferin gene expression,” Genes and Dev., 3:760, 1989.

Moreau, Hen, Wasylyk, Everett, Gaub, and Chambon, “The SV40 base-repair repeat has a striking effect on gene expression both in sv40 and other chimeric recombinants,” Nucl. Acids Res., 9:6047, 1981.

Moriuchi, Oligino, Krisky, Marconi, Fink, Cohen, Glorioso, “Enhanced tumor cell killing in the presence of ganciclovir by herpes simplex virus type 1 vector-directed coexpression of human tumor necrosis factor-alpha and herpes simplex virus thymidine kinase,” Cancer Res, 58(24):5731-5737, 1998.

Morrison, Onions, Nicolson, “Complete DNA sequence of canine adenovirus type 1,” J Gen Virol, 78(Pt 4):873-8, 1997.

Musesing, Smith, and Capon, “Regulation of mRNA Accumulation by a Human Immunodeficiency Virus Trans-Activator Protein,” Cell, 48:691, 1987.

Naldini, Blomer, Gallay, Ory, Mulligan, Gage, Verma, Trono, “In vivo gene delivery and stable transduction of nondividing cells by a lentiviral vector,” Science, 272(5259):263-267, 1996.

Neumann, Watanabe, Ito, Watanabe, Goto, Gao, Hughes, Perez, Donis, Hoffmann, Hobom, Kawaoka, “Generation of influenza A viruses entirely from cloned cDNAs,” Proc Natl Acad Sci USA, 96(16):9345-9350, 1999.

Ng, Gunning, Liu, Leavitt, and Kedes, “Regulation of the Human Beta-Actin Promoter by Upstream and Intron Domains,” Nuc. Acids Res., 17:601, 1989.

Nicolau and Sene, “Liposome-mediated DNA transfer in eukaryotic cells. Dependence of the transfer efficiency upon the type of liposomes used and the host cell cycle stage,” Biochim. Biophys. Acta, 721:185-190, 1982.

Nicolau, Legrand, Grosse, “Liposomes as carriers for in vivo gene transfer and expression,” Methods Enzymol., 149:157-176, 1987.

Nigro, Takahashi, Ginzinger, Law, Passe, Jenkins, Aldape, “Detection of 1p and 19q loss in oligodendroglioma by quantitative microsatellite analysis, a real-time quantitative polymerase chain reaction assay,” Am J Pathol, 158(4):1253-1262, 2001.

Nomoto, Tatematsu, Takahashi, Osada, “Cloning and characterization of the alternative promoter regions of the human LIMK2 gene responsible for alternative transcripts with tissue-specific expression,” Gene, 236(2):259-71, 1999.

Ogunbiyi, Goodfellow, Gagliardi, Swanson, Birnbaum, Fleshman, Kodner, Moley, “Prognostic value of chromosome 1p allelic loss in colon cancer,” Gastroenterology, 113(3):761-766, 1997.

Oh, Muller, Lamson, Rosenfeld, “Insulin-like growth factor (IGF)-independent action of IGF-binding protein-3 in Hs578T human breast cancer cells. Cell surface binding and growth inhibition,” J Biol Chem, 268(20):14964-14971, 1993.

Ohara, Dorit, Gilbert, “One-sided polymerase chain reaction: the amplification of cDNA,” Proc. Nat'l Acad. Sci. USA, 86: 5673-5677, 1989.

Omirulleh, Abraham, Golovkin, Stefanov, Karabaev, Mustardy, Morocz, Dudits, “Activity of a chimeric promoter with the doubled CaMV 35S enhancer element in protoplast-derived cells and transgenic plants in maize,” Plant Mol Biol, 21(3):415-28, 1993.

Omitz, Hammer, Davison, Brinster, and Palmiter, “Promoter and enhancer elements from the rat elastase i gene function independently of each other and of heterologous enhancers,” Mol. Cell. Biol. 7:3466, 1987.

Ondek, Sheppard, and Herr, “Discrete Elements Within the SV40 Enhancer Region Display Different Cell-Specific Enhancer Activities,” EMBO J., 6:1017, 1987.

Parks and Graham, “A helper-dependent system for adenovirus vector producing helps define a lower limit for efficient DNA packaging,” J. Virol., 71(4):3293-3298, 1997.

Palmiter, Chen, and Brinster, “Differential regulation of metallothionein-thymidine kinase fusion genes in transgenic mice and their offspring,” Cell, 29:701, 1982.

PCT Application No. PCT/US87/00880

PCT Application No. PCT/US89/01025

PCT Application No. WO 88/10315

PCT Application No. WO 94/09699

PCT Application No. WO 90/07641

PCT Application No. WO 89/06700

PCT Application No. WO 95/06128

Pease, Solas, Sullivan, Cronin, Holmes, Fodor, “Light-generated oligonucleotide arrays for rapid DNA sequence analysis,” Proc. Natl. Acad. Sci. USA, 91:5022-5026, 1994.

Pech, Rao, Robbins, and Aaronson, “Functional identification of regulatory elements within the promoter region of platelet-derived growth factor 2,” Mol. Cell. Biol., 9:396, 1989.

Pelletier and Sonenberg, “Internal initiation of translation of eukaryotic mRNA directed by a sequence derived from poliovirus RNA,” Nature, 334(6180):320-325, 1988.

Perales, Ferkol, Beegen, Ratnoff, Hanson, “Gene transfer in vivo: sustained expression and regulation of genes introduced into the liver by receptor-targeted uptake,” Proc. Nat'l Acad. Sci. 91:4086-4090, 1994.

Perez-Stable and Constantini, “Roles of fetal y-globin promoter elements and the adult β-globin 3′ enhancer in the stage-specific expression of globin genes,” Mol. Cell. Biol., 10:1116, 1990.

Petrof, “Respiratory muscles as a target for adenovirus-mediated gene therapy,” Eur Respir J, 11(2):492-497, 1998.

Picard and Schaffner, “A lymphocyte-specific enhancer in the mouse immunoglobulin kappa gene,” Nature, 307:83, 1984.

Pignon, Vinatier, Fanen, Jonveaux, Tournilhac, Imbert, Rochant, Goossens, “Exhaustive analysis of the P53 gene coding sequence by denaturing gradient gel electrophoresis: application to the detection of point mutations in acute leukemias,” Hum. Mutat., 3: 126-132, 1994.

Pinkert, Omitz, Brinster, and Palmiter, “An albumin enhancer located 10 kb upstream functions along with its promoter to direct efficient, liver-specific expression in transgenic mice,” Genes and Dev., 1:268, 1987.

Ponta, Kennedy, Skroch, Hynes, and Groner, “Hormonal Response Region in the Mouse Mammary Tumor Virus Long Terminal Repeat Can Be Dissociated From the Proviral Promoter and Has Enhancer Properties,” Proc. Natl. Acad. Sci. U.S.A., 82:1020, 1985.

Porton, Zaller, Lieberson, and Eckhardt, “Immunoglobulin heavy-chain enhancer is required to maintain transfected .gamma.2a gene expression in a pre-b-cell line,” Mol. Cell. Biol., 10:1076, 1990.

Porton, Zaller, Lieberson, and Eckhardt, “Immunoglobulin heavy-chain enhancer is required to maintain transfected .gamma.2a gene expression in a pre-b-cell line,” Mol. Cell. Biol., 10:1076, 1990.

Potrykus, Paszkowski, Saul, Petruska, Shillito, “Molecular and general genetics of a hybrid foreign gene introduced into tobacco by direct gene transfer,” Mol Gen Genet, 199(2):169-77, 1985.

Potter, Weir, Leder, “Enhancer-dependent expression of human kappa immunoglobulin genes introduced into mouse pre-B lymphocytes by electroporation,” Proc. Nat'l Acad. Sci. USA, 81:7161-7165, 1984.

Queen and Baltimore, “Immunoglobulin gene transcription is activated by downstream sequence elements,” Cell, 35:741, 1983.

Quinn, Farina, Gardner, Krutzsch, and Levens, “Multiple components are required for sequence recognition of the ap1 site in the gibbon ape leukemia virus enhancer,” Mol. Cell. Biol., 9:4713, 1989.

Rabinovitch, Suarez-Pinzon, Strynadka, Ju, Edelstein, Brownleee, Korbutt, Rajotte, “Transfection of human pancreatic islets with an anti-apoptotic gene (bc1-2) protects beta-cells from cytokine-induced destruction,” Diabetes, 48(6):1223-1229, 1999.

Ragnarsson, Eiriksdottir, Johannsdottir, Jonasson, Egilsson, Ingvarsson, “Loss of heterozygosity at chromosome 1p in different solid human tumours: association with survival,” Br J Cancer, 79(9-10):1468-1474, 1999.

Rasio, Murakumo, Robbins, Roth, Silver, Negrini, Schmidt, Burczak, Fishel, Croce, “Characterization of the human homologue of RAD54: a gene located on chromosome 1p32 at a region of high loss of heterozygosity in breast tumors,” Cancer Res, 57(12):2378-2383, 1997.

Reddy, Idamakanti, Zakhartchouk, Baxi, Lee, Pyne, Babiuk, Tikoo, “Nucleotide sequence, genome organization and transcription map of bovine adenovirus type 3,” J. Virol., 72(2):1394-1402, 1998.

Redondo, Hata, Brocklehurst, and Krangel, “A T-cell-specific transcriptional enhancer within the human T-cell receptor .delta locus,” Science, 247:1225, 1990.

Reifenberger, Reifenberger, Liu, James, Wechsler, Collins, “Molecular genetic analysis of oligodendroglial tumors shows preferential allelic deletions on 19q and 1p,” Am J Pathol, 145(5):1175-1190, 1994.

Reinhold-Hurek and Shub, “Self-splicing introns in tRNA genes of widely divergent bacteria,” Nature, 357:173-176, 1992.

Reisman and Rotter, “Induced expression from the moloney murine leukemia virus long terminal repeat during differentiation of human myeloid cells is mediated through its transcriptional enhancer,” Mol. Cell. Biol., 9:3571, 1989.

Remington's Pharmaceutical Sciences, 15th Edition, Chapter 61, pages 1035-1038 and 1570-1580.

Renehan, Jones, Potten, Shalet, O'Dwyer, “Elevated serum insulin-like growth factor (IGF)-II and IGF binding protein-2 in patients with colorectal cancer,” Br J Cancer, 83(10):1344-1350, 2000.

Resendez Jr., Wooden, and Lee, “Identification of highly conserved regulatory domains and protein-binding sites in the promoters of the rat and human genes encoding the stress-inducible 78-kilodalton glucose-regulated protein,” Mol. Cell. Biol., 8:4579, 1988.

Ripe, Lorenzen, Brenner, and Breindl, “Regulatory elements in the 5′ flanking region and the first intron contribute to transcriptional control of the mouse alpha-1-type collagen gene,” Mol. Cell. Biol., 9:2224, 1989.

Rippe, Brenner and Leffert, “DNA-mediated gene transfer into adult rat hepatocytes in primary culture,” Mol. Cell Biol., 10:689-695, 1990.

Rittling, Coutinho, Amarm, and Kolbe, “AP-1/jun-binding sites mediate serum inducibility of the human vimentin promoter,” Nuc. Acids Res., 17:1619, 1989.

Robbins and Ghivizzani, “Viral vectors for gene therapy,” Pharmacol Ther, 80(1):35-47, 1998.

Robbins, Tahara, Ghivizzani, “Viral vectors for gene therapy,” Trends Biotechnol, 16(1):35-40, 1998.

Rosen, Sodroski, and Haseltine, “The location of cis-acting regulatory sequences in the human t-cell lymphotropic virus type III (HTLV-111/LAV) long terminal repeat,” Cell, 41:813, 1988.

Sakai, Helms, Carlstedt-Duke, Gustafsson, Rottman, and Yamamoto, “Hormone-Mediated Repression: A Negative Glucocorticoid-Response Element From the Bovine Prolactin Gene,” Genes and Dev., 2:1144,1988.

Sambrook et. al., In: Molecular Cloning: A Laboratory Manual, 2d Ed., Cold Spring Harbor Laboratory Press, Cold Spring Harbor, N.Y., 1989.

Sarver, Cantin, Chang, Zaia, Ladne, Stephens, Rossi, “Ribozymes as potential anti-HIV-1 therapeutic agents,” Science, 247:1222-1225, 1990.

Satake, Furukawa, and Ito, “Biological activities of oligonucleotides spanning the f9 point mutation within the enhancer region of polyoma virus DNA,” J. Virology, 62:970, 1988.

Sawai, Ikeda, Ishizu, Meruelo, “Reducing cytotoxicity induced by Sindbis viral vectors,” Mol Genet Metab, 67(1):36-42, 1999.

Scanlon, Jiao, Funato, Wang, Tone, Rossi, Kashani-Sabet, “Ribozyme-mediated cleavages of c-fos mRNA reduce gene expression of DNA synthesis enzymes and metallothionein,” Proc Natl Acad Sci USA, 88:10591-10595, 1991.

Schaffner, Schirm, Muller-Baden, Wever, and Schaffner, “Redundancy of Information in Enhancers as a Principle of Mammalian Transcription Control,” J. Mol. Biol., 201:81, 1988.

Searle, Stuart, and Palmiter, “Building a metal-responsive promoter with synthetic regulatory elements,” Mol. Cell. Biol., 5:1480, 1985.

Sharp and Marciniak, “HIV Tar: an RNA Enhancer?,” Cell, 59:229, 1989.

Shaul and Ben-Levy, “Multiple Nuclear Proteins in Liver Cells are Bound to Hepatitis B Virus Enhancer Element and its Upstream Sequences,” EMBO J., 6:1913, 1987.

Sherman, Basta, Moore, Brown, and Ting, “Class II Box Consensus Sequences in the HLA-DR.alpha. Gene: Transcriptional Function and Interaction with Nuclear Proteins,” Mol. Cell. Biol.,9:50, 1989.

Shoemaker, Lashkari, Morris, Mittmann, Davis, “Quantitative phenotypic analysis of yeast deletion mutants using a highly parallel molecular bar-coding strategy,” , Nature Genetics 14:450-456, 1996.

Shub and Goodrich-Blair, “Protein introns: a new home for endonucleases,” Cell, 71(2):183-186, 1992.

Sleigh and Lockett, “SV40 Enhancer Activation During Retinoic-Acid-Induced Differentiation of F9 Embryonal Carcinoma Cells,” J. EMBO, 4:3831, 1985.

Smith, Perry, Borell, Lee, O'Fallon, Hosek, Kimmel, Yates, Burger, Scheithauer, Jenkins, “Alterations of chromosome arms 1p and 19q as predictors of survival in oligodendrogliomas, astrocytomas, and mixed oligoastrocytomas,” J Clin Oncol, 18(3):636-45, 2000.

Spalholz, Yang, and Howley, “Transactivation of a Bovine Papilloma Virus Transcriptional Regulatory Element by the E2 Gene Product,” Cell, 42:183, 1985.

Spandau and Lee, “Trans-Activation of Viral Enhancers by the Hepatitis B Virus X Protein,” J. Virology, 62:427, 1988.

Spandidos and Wilkie, “Host-Specificities of Papilloma Virus, Moloney Murine Sarcoma Virus and Simian Virus 40 Enhancer Sequences,” EMBO J., 2:1193, 1983.

Stephens and Hentschel, “The Bovine Papilloma Virus Genome and its Uses as a Eukaryotic Vector,” Biochem. J., 248:1, 1987.

Stewart and Young, Solid Phase Peptide Synthesis, 2d. ed., Pierce Chemical Co., 1984.

Stewart, Lassam, Quirt, Bailey, Rotstein, Krajden, Dessureault, Gallinger, Cappe, Wan, Addison, Moen, Gauldie, Graham, “Adenovector-mediated gene delivery of interleukin-2 in metastatic breast cancer and melanoma: results of a phase 1 clinical trial,” Gene Ther, 6(3):350-363, 1999.

Stuart, Searle, and Palmiter, “Identification of Multiple Metal Regulatory Elements in Mouse Metallothionein-I Promoter by Assaying Synthetic Sequences,” Nature, 317:828, 1985.

Sullivan and Peterlin, “Transcriptional enhancers in the HLA-DQ subregion,” Mol. Cell. Biol., 7:3315, 1987.

Suzuki, Piche, Kasono, Xiang, Gomez-Navarro, Moriuchi, Krisky, Oligino, Glorioso, Curiel, Curiel, “Efficient gene delivery into epstein-barr virus (EBV)-transformed human B cells mediated by replication-defective herpes simplex virus-1 (HSV-1): A gene therapy model for EBV-related B cell malignancy,” Biochem Biophys Res Commun, 252(3):686-90, 1998.

Swartzendruber and Lehman, “Neoplastic Differentiation: Interaction of Simian Virus 40 and Polyoma Virus with Murine Teratocarcinoma Cells,” J. Cell. Physiology, 85:179, 1975.

Takebe, Seiki, Fujisawa, Hoy, Yokota, Arai, Yoshida, and Arai, “SRα promoter: an efficient and versatile mammalian cDNA expression system composed of the simian virus 40 early promoter and the R-U5 segment of human T-cell leukemia virus type 1 long terminal repeat,” Mol. Cell. Biol., 8:466, 1988.

Tam et al., J. Am. Chem. Soc., 105:6442, 1983.

Tanaka, Yanoshita, Konishi, Oshimura, Maeda, Mori, Miyaki, “Suppression of tumourigenicity in human colon carcinoma cells by introduction of normal chromosome 1p36 region,” Oncogene, 8(8):2253-2258, 1993.

Tavernier, Gheysen, Duerinck, Can Der Heyden, and Fiers, “Deletion mapping of the inducible promoter of human IFN-beta gene,” Nature, 301:634, 1983.

Taylor and Kingston, “E1A Trans-Activation of Human HSP70 Gene Promoter Substitution Mutants is Independent of the Composition of Upstream and TATA Elements,” Mol. Cell. Biol., 10:176, 1990b.

Taylor and Kingston, “Factor Substitution in a Human HSP70 Gene Promoter: TATA-Dependent and TATA-Independent Interactions,” Mol. Cell. Biol., 10:165, 1990a.

Taylor, Solomon, Weiner, Paucha, Bradley, and Kingston, “Stimulation of the Human Heat-Shock Protein 70 Promoter in vitro by Simian Virus 40 Large T Antigen,” J. Biol. Chem., 264:15160, 1989.

Thiesen, Bosze, Henry, and Charnay, “A DNA Element Responsible for the Different Tissue Specificities of Friend and Moloney Retroviral Enhancers,” J. Virology, 62:614, 1988.

Timiryasova T M, Chen B, Haghighat P, Fodor, “Vaccinia virus-mediated expression of wild-type p53 suppresses glioma cell growth and induces apoptosis,” Int J Oncol, 14(5):845-854, 1999.

Treisman, “Transient Accumulation of c-fos RNA Following Serum Stimulation Requires a Conserved 5′ Element and c-fos 3′ Sequences,” Cell, 42:889, 1985.

Tronche, Rollier, Bach, Weiss, and Yaniv, “The Rat Albumin Promoter: Cooperation with Upstream Elements is Required When Binding of APF/HNF 1 to the Proximal Element is Partially Impaired by Mutation or Bacterial Methylation,” Mol. Cell. Biol., 9:4759, 1989.

Tronche, Rollier, Herbomel, Bach, Cereghini, Weiss, and Yaniv, “Anatomy of the Rat Albumin Promoter,” Mol. Biol. Med., 7:173, 1990.

Trudel and Constantini, “A 3′ Enhancer Contributes to the Stage-Specific Expression of the Human Beta-Globin Gene,” Genes and Dev., 6:954, 1987.

Tsumaki et al., “Modular arrangement of cartilage- and neural tissue-specific cis-elements in the mouse alpha2(XI) collagen promoter,” J Biol Chem. 273(36):22861-22864, 1998.

Tur-Kaspa, Teicher, Levine, Skoultchi and Shafritz, “Use of electroporation to introduce biologically active foreign genes into primary rat hepatocytes,” Mol. Cell Biol., 6:716-718, 1986.

Tyndall, La Mantia, Thacker, Favaloro, and Kamen, “A Region of the Polyoma Virus Genome Between the Replication Origin and Late Protein-Coding Sequences is Required in cis for Both Early Gene Expression and Viral DNA Replication,” Nuc. Acids. Res., 9:6231, 1981.

Vanderkwaak, Wang, Gomez-Navarro, Rancourt, Dmitriev, Krasnykh, Barnes, Siegal, Alvarez, Curiel, “An advanced generation of adenoviral vectors selectively enhances gene transfer for ovarian cancer gene therapy approaches,” Gynecol Oncol, 74(2):227-234, 1999.

Vannice and Levinson, “Properties of the Human Hepatitis B Virus Enhancer: Position Effects and Cell-Type Nonspecificity,” J. Virology, 62:1305, 1988.

Vasseur, Kress, Montreau, and Blangy, “Isolation and Characterization of Polyoma Virus Mutants Able to Develop in Multipotential Murine Embryonal Carcinoma Cells,” Proc Natl. Acad. Sci. U.S.A., 77:1068, 1980.

Wagner, Zenke, Cotten, Beug, Birnstiel, “Transferrin-polycation conjugates as carriers for DNA uptake into cells,” Proc. Nat'l Acad. Sci. USA 87(9):3410-3414, 1990.

Walker, Fraiser, Schram, Little, Nadeau, Malinowski, “Strand displacement amplification—an isothermal, in vitro DNA amplification technique,” Nucleic Acids Res. 20(7):1691-1696, 1992.

Wang and Calame, “SV40 enhancer-binding factors are required at the establishment but not the maintenance step of enhancer-dependent transcriptional activation,” Cell, 47:241, 1986.

Weber, De Villiers, and Schaffner, “An SV40 Enhancer Trap Incorporates Exogenous Enhancers or Generates Enhancers From its Own Sequences,” Cell, 36:983, 1984.

Weihl, Macdonald, Stoodley, Luders, Lin, “Gene therapy for cerebrovascular disease,” Neurosurgery, 44(2):239-252, 1999.

Weinberg, “The molecular basis of retinoblastomas,” Ciba Found Symp., 142:99-105, 1989.

Weinberger, Jat, and Sharp, “Localization of a Repressive Sequence Contributing to B-cell Specificity in the Immunoglobulin Heavy-Chain Enhancer,” Mol. Cell. Biol., 8:988, 1984.

Wex, Vorwerk, Mohnike, Bretschneider, Kluba, Aumann, Blum, Mittler, “Elevated serum levels of IGFBP-2 found in children suffering from acute leukaemia is accompanied by the occurrence of IGFBP-2 mRNA in the tumour clone,” Br J Cancer, 78(4):515-520, 1998.

Wilson, “When bad gene transfer is good,” J Clin Invest, 98(11):2435, 1996.

Winoto and Baltimore, “αβ-lineage-specific Expression of the α T-Cell Receptor Gene by Nearby Silencers,” Cell, 59:649, 1989.

Wong, Nicolau, Hofschneider, “Appearance of β-lactamase activity in animal cells upon liposome mediated gene transfer,” Gene, 10:87-94, 1980.

Wu and Wu, “Receptor-mediated in vitro gene transfections by a soluble DNA carrier system,” J. Biol. Chem., 262:4429-4432, 1987.

Wu and Wu, Adv. Drug Delivery Rev., 12:159-167, 1993.

Wu, “Recent advances in gene therapy of GI and liver diseases,” Zhonghua Min Guo Xiao Er Ke Yi Xue Hui Za Zhi, September-October, 39(5):297-300, 1998.

Wu, Squire, Song, Weksberg, “Promoter-dependent tissue-specific expressive nature of imprinting gene, insulin-like growth factor II, in human tissues,” Biochem Biophys Res Commun. 233(1):221-226, 1997.

Yamada, Oligino, Mata, Goss, Glorioso, Fink, “Herpes simplex virus vector-mediated expression of Bcl-2 prevents 6-hydroxydopamine-induced degeneration of neurons in the substantia nigra in vivo,” Proc Natl Acad Sci USA, 96(7):4078-4083, 1999.

Yang, Burkholder, Roberts, Martinell and McCabe, “In vivo and in vitro gene transfer to mammalian somatic cells by particle bombardment,” Proc Nat'l Acad Sci. USA, 87:9568-9572, 1990.

Yeung, Bockhold, Tufaro, “Efficient infection of mature skeletal muscle with herpes simplex virus vectors by using dextran sulfate as a co-receptor,” Gene Ther., 6(9):1536-1544, 1999.

Yoon, Carroll, Chiocca, Tanabe, “Influence of p53 on herpes simplex virus type 1 vectors for cancer gene therapy,” J. Gastrointest. Surg., 3(1):34-48, 1999.

Yutzey, Kline, and Konieczny, “An Internal Regulatory Element Controls Troponin I Gene Expression,” Mol. Cell. Biol., 9:1397, 1989.

Zhao-Emonet, Boyer, Cohen, Klatzmann, “Deletional and mutational analyses of the human CD4 gene promoter: characterization of a minimal tissue-specific promoter,” Biochim Biophys Acta, 1442(2-3):109-119, 1998.

Zheng, Graham, Prevec, “Transcription units of E1a, E1b and pIX regions of bovine adenovirus type 3,” J Gen Virol, 80 (Pt 7):1735-1742, 1999.

Zufferey, Nagy, Mandel, Naldini, Trono, “Multiply attenuated lentiviral vector achieves efficient gene delivery in vivo,” Nat Biotechnol, 15(9):871-875, 1997.

What is claimed:

1. An isolated polypeptide comprising an amino acid sequence selected from the group consisting of SEQ ID NO: 1, SEQ ID NO: 2 or SEQ ID NO: 3.

2. A polynucleotide sequence encoding a polypeptide having the amino acid sequence of SEQ ID NO: 1, SEQ ID NO: 2 or SEQ ID NO: 3.

3. An isolated polynucleotide comprising a nucleotide sequence selected from the group consisting of SEQ ID NO: 4, SEQ ID NO: 5 or SEQ ID NO: 6.

4. An expression cassette comprising a polynucleotide encoding a polypeptide having the amino acid sequence of SEQ ID NO: 1, SEQ ID NO: 2 or SEQ ID NO: 3, wherein said polynucleotide is under the control of a promoter operable in eukaryotic cells.

5. The expression cassette of claim 4, wherein said promoter is heterologous to the coding sequence.

6. The expression cassette of claim 5, wherein said promoter is selected from the group consisting of Hsp68, SV36, CMV, MKC, GAL4_{UAS}, HSV and β-actin.

7. The expression cassette of claim 5, wherein said promoter is a tissue specific promoter.

8. The expression cassette of claim 5, wherein said promoter is an inducible promoter.

9. The expression cassette of claim 5, wherein said expression cassette is contained in a viral vector.

10. The expression cassette of claim 5, wherein said viral vector is selected from the group consisting of a retroviral vector, an adenoviral vector, and adeno-associated viral vector, a vaccinia viral vector, and a herpesviral vector.

11. The expression cassette of claim 4, wherein said expression cassette further comprises a polyadenylation signal.

12. The expression cassette of claim 4, wherein said expression cassette comprises a second polynucleotide encoding a second polypeptide.

13. The expression cassette of claim 12, wherein said second polynucleotide is under the control of a second promoter.

14. A method for expressing a polypeptide comprising contacting said cells with an expression cassette comprising a polynucleotide encoding a polypeptide having the amino acid sequence of SEQ ID NO: 1, SEQ ID NO: 2 or SEQ ID NO: 3, wherein said polynucleotide is under the control of a promoter operable in eukaryotic cells.

15. The method of claim 14, wherein said promoter is heterologous to the polynucleotide sequence.

16. The method of claim 15, wherein said promoter is selected from the group consisting of Hsp68, SV36, CMV, MKC, GAL4_{UAS}, HSV and β-actin.

17. The method of claim 15, wherein said promoter is a constitutive promoter.

18. The method of claim 15, wherein said promoter is an inducible promoter.

19. The method of claim 15, wherein said expression cassette is contained in a viral vector.

20. The method of claim 19, wherein said viral vector is selected from the group consisting of a retroviral vector, an adenoviral vector, an adeno-associated viral vector, a vaccinia viral vector, and a herpesviral vector.

21. The method of claim 14, wherein said expression cassette further comprises a polyadenylation signal.

22. The method of claim 14, wherein said expression cassette comprises a second polynucleotide encoding a second polypeptide.

23. The method of claim 22, wherein said second polynucleotide is under the control of a second promoter.

24. A host cell comprising an expression cassette comprising a polynucleotide encoding a polypeptide having the amino acid sequence of SEQ ID NO: 1, SEQ ID NO: 2 or SEQ ID NO: 3, wherein said polynucleotide is under the control of a heterologous promoter operable in eukaryotic cells.

25. A method of diagnosing cancer comprising the steps of:
(a) obtaining a sample from a subject; and
(b) characterizing the expression of a polypeptide having the amino acid sequence SEQ ID NO: 1, SEQ ID NO: 2 or SEQ ID NO: 3.

26. The method of claim 25, wherein said cancer is selected from the group consisting of brain, lung, liver, spleen, kidney, lymph node, small intestine, pancreas, blood cells, colon, stomach, breast, endometrium, prostate, testicle, ovary, skin, head and neck, esophagus, bone marrow and blood cancer.

27. The method of claim 26, wherein said cancer is brain cancer.

28. The method of claim 26, wherein said brain cancer is a glioblastoma.

29. The method of claim 26, wherein said sample is a tissue or fluid sample.

30. The method of claim 25, wherein said assessing comprises assaying for a nucleotide sequence.

31. The method of claim 30, wherein said nucleotide sequence is a mRNA, a cDNA or a genomic DNA.

32. The method of claim 30, further comprising subjecting said sample to conditions suitable to nucleic acid amplification.

33. The method of claim 30, wherein assaying comprises evaluating the level of expression of said nucleotide sequence.

34. The method of claim 30, further comprising the step of comparing the expression of said nucleotide sequences with the expression of said nucleotide sequences in non-cancer samples.

35. The method of claim 30, wherein assaying involves evaluating the structure of said nucleotide sequence.

36. The method of claim 35, wherein said evaluating comprises an assay selected from the group consisting of sequencing, wild-type oligonucleotide hybridization, mutant oligonucleotide hybridization, SSCP, PCR and RNase protection.

37. The method of claim 36, wherein said wild-type or mutant oligonucleotide hybridization employs one or more oligonucleotides configured in an array on a chip or wafer.

38. The method of claim 25, wherein said assessing comprises assaying for a polypeptide sequence.

39. The method of claim 38, wherein assaying comprises immunologic detection.

40. The method of claim 38, wherein assaying comprises evaluating the level of expression of said polypeptide sequence.

41. The method of claim 38, wherein assaying involves evaluating the structure of said polypeptide sequence.

42. A method for altering the phenotype of a tumor cell comprising the step of administering to a cell a polypeptide having the amino acid sequence SEQ ID NO: 1 under conditions permitting the uptake of said polypeptide by said tumor cell.

43. The method of claim 42, wherein said tumor cell is derived from a tissue selected from the group consisting of brain, lung, liver, spleen, kidney, lymph node, small intestine, blood cells, pancreas, colon, stomach, breast, endometrium, prostate, testicle, ovary, skin, head and neck, esophagus, bone marrow and blood tissue.

44. The method of claim 43, wherein the phenotype is selected from the group consisting of apoptosis, angiogenesis, proliferation, migration, contact inhibition, soft agar growth and cell cycling.

45. The method of claim 43, wherein said polypeptide is encapsulated in a liposome.

46. A method for altering the phenotype of a tumor cell comprising the step of contacting said cell with a nucleic acid comprising (i) a region encoding a polypeptide having the amino acid sequence SEQ ID NO: 1 and (ii) a promoter active in said tumor cell, wherein said promoter is operably linked to said encoding region, under conditions permitting the uptake of said nucleic acid by said tumor cell.

47. The method of claim 46, wherein said tumor cell is derived from a tissue selected from the group consisting of brain, lung, liver, spleen, kidney, lymph node, small intestine, blood cells, pancreas, colon, stomach, breast, endometrium, prostate, testicle, ovary, skin, head and neck, esophagus, bone marrow and blood tissue.

48. The method of claim 46, wherein the a phenotype is selected from the group consisting of apoptosis, angiogenesis, proliferation, migration, contact inhibition, soft agar growth or cell cycling.

49. The method of claim 46, wherein said nucleic acid is encapsulated in a liposome.

50. The method of claim 46, wherein said nucleic acid is a viral vector selected from the group consisting of retrovirus, adenovirus, adeno-associated virus, vaccinia virus and herpesvirus.

51. The method of claim 50, wherein said nucleic acid is encapsulated in a viral particle.

52. A method for treating a subject with cancer comprising the step of administering to said subject a polypeptide having the amino acid sequence SEQ ID NO: 1.

53. The method of claim 52, wherein said tumor cell is derived from a tissue selected from the group consisting of brain, lung, liver, spleen, kidney, lymph node, small intestine, blood cells, pancreas, colon, stomach, breast, endometrium, prostate, testicle, ovary, skin, head and neck, esophagus, bone marrow and blood tissue.

54. The method of claim 52, wherein the subject is a human.

55. A method for treating a subject with cancer comprising the step of administering to said subject a nucleic acid comprising (i) a region encoding a polypeptide having the amino acid sequence SEQ ID NO: 1 and (ii) a promoter active in said tumor cell, wherein said promoter is operably linked to said encoding region.

56. The method of claim 55, wherein said tumor cell is derived from a tissue selected from the group consisting of brain, lung, liver, spleen, kidney, lymph node, small intestine, blood cells, pancreas, colon, stomach, breast, endometrium, prostate, testicle, ovary, skin, head and neck, esophagus, bone marrow and blood tissue.

57. The method of claim 50, wherein the subject is a human.

58. A non-human transgenic eukaryote comprising at least one non-functional 86F allele.

59. The non-human transgenic eukaryote of claim 58, comprising two non-functional 86F alleles.

60. The non-human transgenic eukaryote of claim 58, wherein said eukaryote is a mouse.

61. A non-human transgenic eukaryote that overexpresses 86F as compared to a similar non-transgenic eukaryote.

62. A non-human transgenic eukaryote that overexpresses 86S as compared to a similar non-transgenic eukaryote.

63. A non-human transgenic eukaryote that overexpresses 86Y as compared to a similar non-transgenic eukaryote.

64. A method of screening a candidate substance for anti-tumor activity comprising the steps of:
(i) providing a cell lacking a functional 86F polypeptide;
(ii) contacting said cell with said candidate substance; and
(iii) determining the effect of said candidate substance on said cell.

65. The method of claim 64, wherein said cell is a tumor cell.

66. The method of claim 64, wherein said tumor cell has a mutation in the coding region of 86F.

67. The method of claim 64, wherein said tumor cell has a mutation in the non-coding regulatory region of 86F.

68. The method of claim 66, wherein said mutation is a deletion mutant, an insertion mutant, a frameshift mutant, a nonsense mutant, a missense mutant or splice mutant.

69. The method of claim 64, wherein said determining comprises comparing one or more characteristics of the cell in the presence of said candidate substance with characteristics of a cell in the absence of said candidate substance.

70. The method of claim 69, wherein said characteristic is selected from the group consisting of 86 expression, IGFBP2 expression, phosphatase activity, proliferation, metastasis, contact inhibition, soft agar growth, cell cycle regulation, tumor formation, tumor progression and tissue invasion.

71. The method of claim 64, wherein said candidate substance is a chemotherapeutic agent, a radiotherapeutic agent or is selected from a small molecule library.

72. The method of claim 64, wherein said cell is contacted in vitro.

73. The method of claim 64, wherein said cell in contacted in vivo.

74. A method of screening a candidate substance for anti-tumor activity comprising the steps of:
(i) providing a cell expressing the 86S polypeptide;
(ii) contacting said cell with said candidate substance; and
(iii) determining the effect of said candidate substance on said cell.

75. The method of claim 74, wherein said cell is a tumor cell.

76. The method of claim 74, wherein said determining comprises comparing one or more characteristics of the cell in the presence of said candidate substance with characteristics of a cell in the absence of said candidate substance.

77. The method of claim 76, wherein said characteristic is selected from the group consisting of 86 expression, IGFBP2 expression, phosphatase activity, proliferation, metastasis, contact inhibition, soft agar growth, cell cycle regulation, tumor formation, tumor progression and tissue invasion.

78. The method of claim 76, wherein said candidate substance is a chemotherapeutic agent, a radiotherapeutic agent or is selected from a small molecule library.

79. The method of claim 76, wherein said cell is contacted in vitro.

80. The method of claim 76, wherein said cell in contacted in vivo.

81. A method of screening a candidate substance for the ability to modulate the protein-protein interaction between 86 and IGFBP2 comprising the steps of:
(i) providing a cell expressing 86 and IGFBP2;
(ii) contacting said cell with said candidate substance; and
(iii) determining the effect of said candidate substance on said protein-protein interaction.

82. A monoclonal antibody that binds immunologically to an epitope common between 86F and 86S.

83. A monoclonal antibody that binds immunologically to an epitope common between 86F, 6S and 86Y.

84. A monoclonal antibody that binds immunologically to 86F but not 86S or 86Y.

85. A monoclonal antibody that binds immunologically to 86S but not 86F or 86Y.

86. A monoclonal antibody that binds immunolgically to 86Y but not to 86F or 86S.

87. A polyclonal antisera that binds immunologically to an 86 polypeptide.


SEQUENCE LISTING (8 sequences)

SEQ ID NO: 1 - protein, 388 aa, Artificial Sequence
  Description of Artificial Sequence  Synthetic Peptide
MVEAEELAQLRLLNLELLRQLWVGQDAVRRSVARAASESSLESSSSYNSETPSTPETSSTSLSTSCPRGRSSVWGPPDACRGDLRDVARSGVASLPPAKCQHQESLGRPRPHSAPSLGTSSLRDPEPSGRLGDPGPQEAQTPRSILAQQSKLSKPRVTFSEESAVPERSWRLRPYLGYDWIAGSLDTSSSITSQPEAFFSKLQEFRETNKEECICSHPEPQLPGLRESSGSGVEEDHECVYCYRVNRRLFPVPVDPGTPCRLCRTPRDQQGPGTLAQPAHVRVSIPLSILEPPHRYHIHRRKSFDASDTLALPRHCLLGWDIFPPKSEKSSAPRNLDLWSSVSAEAQHQKLSGTSSPFHPASPMQMLPPTPTWSVPQVPRPHVPRQKP

SEQ ID NO: 2 - protein, 358 aa, Artificial Sequence
  Description of Artificial Sequence  Synthetic Peptide
MVEAEELAQLRLLNLELLRQLWVGQDAVRRSVARAASESSLESSSSYNSETPSTPETSSTSLSTSCPRGRSSVWGPPDACRGDLRDVARSGVASLPPAKCQHQESLGRPRPHSAPSLGTSSLRDPEPSGRLGDPGPQEAQTPRSILAQQSKLSKPRVTFSEESAVPKRSWRLRPYLGYDWIAGSLDTSSSITSQPEAFFSKLQEFRETNKEECICSHPEPQLPGLRESSGSGVEEDHECVYCYRVNRRLFPVPVDPGTPCRLCRTPRDQQGPGTLAQPAHVSTACWAGTFFLRSLRKAQPPGTWTSGPLYPLRPSTRSCPAPAALFTRPHQCRCCPRPRPGQCPRSLGPTSHGRSPED

SEQ ID NO: 3 - protein, 366 aa, Artificial Sequence
  Description of Artificial Sequence  Synthetic Peptide
MVEAEELAQLRLLNLELLRQLWVGQDAVRRSVARAASESSLESSSSYNSETPSTPETSSTSLSTSCPRGRSSVWGPPDACRGDLRDVARSGVASLPPAKCQHQESLGRPRPHSAPSLGTSSLRDPEPSGRLGDPGPQEAQTPRSILAQQSKLSKPRVTFSEESAVPERSWRLRPYLGYDWIAGSLDTSSSITSQPEAFFSKLQEFRETNKEECICSHPEPQLPGLRESSGSGVEEDHECVYCYRVNRRLFPVPVDPGTPCRLCRTPRDQQGPGTLAQPAHVRVSIPLSILEPPHRYHIHRRKSFDASDTLALPRHCLLGWDIFPPKSEKSSAPRNLDLWSSVSAEAQHQKLSGTSSPFHPGPALQP

SEQ ID NO: 4 - DNA, 1178 bp, Artificial Sequence
  Description of Artificial Sequence  Synthetic Primer
atggtggaggctgaggaactggcacagctgcggctgctcaatctggagctcctgaggcagctgtgggtggggcaggatgctgtgcggcggtcagtggccagggcagcctcggagtcaagcctggaatccagcagcagctacaactcagagactccatcgaccccagagacgtcctcaacttccttgagcacctcctgcccacggggccggtcctccgtgtggggcccaccagatgcctgtcgaggggacctccgtgatgtggccagatcgggggtggcctctctcccacctgccaagtgccagcaccaggagtccctgggccgaccgagaccccactcagcaccctcgctgggcacctcaagcctgagggacccagagccctcagggaggctgggtgatccaggaccccaggaggcacagaccccgaggtccatcctggctcaacagagcaagctgtccaagcccagggtgaccttctctgaggagtctgcagttcctgagaggagctggcgcctcaggccatacctgggctatgactggattgcagggtctctggacaccagctcttccatcaccagccagcctgaggccttcttctccaagctgcaggagtttcgggaaaccaacaaggaggagtgtatctgcagccatcctgaaccccagttgccaggcctgcgtgagagcagtggcagcggcgtggaggaagaccatgaatgcgtgtactgttaccgtgtcaaccggcgcctgttcccggtgcctgtggatcccggtaccccctgccgcctgtgcaggacaccgcgagaccagcagggccctgggaccctggcgcagccagcgcacgtcagggtgagcatcccgctgtcgatcctggagcccccgcaccggtaccacatccaccggcgaaagagctttgacgcctctgacacactggccctgccccggcactgcctgctgggctgggacatttttcctccgaagtctgagaaaagctcagcccccaggaacctggacctctggtcctctgtctccgctgaggcccagcaccagaagctgtccggcaccagcagcccttttcacccggcctcaccaatgcagatgctgcccccgaccccgacctggtcagtgccccaggtccctcggccccacgtcccacggcagaagccctgaggactgactcc

SEQ ID NO: 5 - DNA, 1081 bp, Artificial Sequence
  Description of Artificial Sequence  Synthetic Primer
atggtggaggctgaggaactggcacagctgcggctgctcaatctggagctcctgaggcagctgtgggtggggcaggatgctgtgcggcggtcagtggccagggcagcctcggagtcaagcctggaatccagcagcagctacaactcagagactccatcgaccccagagacgtcctcaacttccttgagcacctcctgcccacggggccggtcctccgtgtggggcccaccagatgcctgtcgaggggacctccgtgatgtggccagatcgggggtggcctctctcccacctgccaaatgccagcaccaggagtccctgggccgaccgagaccccactcagcaccctcgctgggcacctcaagcctgagggacccagagccctcagggaggctgggtgatccaggaccccaggaggcacagaccccgaggtccatcctggctcaacagagcaagctgtccaagcccagggtgaccttctctgaggagtctgcagttcctaagaggagctggcgcctcaggccatacctgggctatgactggattgcagggtctctggacaccagctcttccatcaccagccagcctgaggccttcttctccaagctgcaggagtttcgggaaaccaacaaggaggagtgtatctgcagccatcctgaaccccagttgccaggcctgcgtgagagcagtggcagcggcgtggaggaagaccatgaatgcgtgtactgttaccgtgtcaaccggcgcctgttcccggtgcctgtggatcccggtaccccctgccgcctgtgcaggacaccgcgagaccagcagggccctgggaccctggcgcagccagcgcacgtcagcactgcctgctgggctgggacatttttcctccgaagtctgagaaaagctcagcccccaggaacctggacctctggtcctctgtatccgctgaggcccagcaccagaagctgtccggcaccagcagcccttttcacccggcctcaccaatgcagatgctgcccccgaccccgacctggtcagtgccccaggtccctcggccccacgtcccacggcagaagccctgaggactgactcc

SEQ ID NO: 6 - DNA, 1361 bp, Artificial Sequence
  Description of Artificial Sequence  Synthetic Primer
atggtggaggctgaggaactggcacagctgcggctgctcaatctggagctcctgaggcagctgtgggtggggcaggatgctgtgcggcggtcagtggccagggcagcctcggagtcaagcctggaatccagcagcagctacaactcagagactccatcgaccccagagacgtcctcaacttccttgagcacctcctgcccacggggccggtcctccgtgtggggcccaccagatgcctgtcgaggggacctccgtgatgtggccagatcgggggtggcctctctcccacctgccaaatgccagcaccaggagtccctgggccgaccgagaccccactcagcaccctcgctgggcacctcaagcctgagggacccagagccctcagggaggctgggtgatccaggaccccaggaggcacagaccccgaggtccatcctggctcaacagagcaagctgtccaagcccagggtgaccttctctgaggagtctgcagttcctgagaggagctggcgcctcaggccatacctgggctatgactggattgcagggtctctggacaccagctcttccatcaccagccagcctgaggccttcttctccaagctgcaggagtttcgggaaaccaacaaggaggagtgtatctgcagccatcctgaaccccagttgccaggcctgcgtgagagcagtggcagcggcgtggaggaagaccatgaatgcgtgtactgttaccgtgtcaaccggcgcctgttcccggtgcctgtggatcccggtaccccctgccgcctgtgcaggacaccgcgagaccagcagggccctgggaccctggcgcagccagcgcacgtcagggtgagcatcccgctgtcgatcctggagcccccgcaccggtaccacatccaccggcgaaagagctttgacgcctctgacacactggccctgccccggcactgcctgctgggctgggacatttttcctccgaagtctgagaaaagctcagcccccaggaacctggacctctggtcctctgtctccgctgaggcccagcaccagaagctgtccggcaccagcagcccttttcacccgggtccagccctccagccctgaccctagccatccctcggaactcctctgctccctgccgcctgccctgctccacccaggaaggccaccctgactgcaagatccaggagggaatgtggctggaactggggatgacctgtccctttcaagtggccccccctgagactttccctattccccatccaggcctcaccaatgcagatgctgcccccgaccccgacctggtcagtgccccaggtccctcggccccacgtcccacggcagaagccctgaggactgactcc

SEQ ID NO: 7 - DNA, 1514 bp, Homo sapiens
  CDS (113)..(1279)
acggcggcgcatgctaggggattctgccgggtagaagagctgggcctggaacccagccctgaggacatcctgcggcccaggggcaagtgacacctgctgagagaggcccaggatggtggaggctgaggaactggcacagctgcggctgctcaatctggagctcctgaggcagctgtgggtggggcaggatgctgtgcggcggtcagtggccagggcagcctcggagtcaagcctggaatccagcagcagctacaactcagagactccatcgaccccagagacgtcctcaacttccttgagcacctcctgcccacggggccggtcctccgtgtggggcccaccagatgcctgtcgaggggacctccgtgatgtggccagatcgggggtggcctctctcccacctgccaagtgccagcaccaggagtccctgggccgaccgagaccccactcagcaccctcgctgggcacctcaagcctgagggacccagagccctcagggaggctgggtgatccaggaccccaggaggcacagaccccgaggtccatcctggctcaacagagcaagctgtccaagcccagggtgaccttctctgaggagtctgcagttcctgagaggagctggcgcctcaggccatacctgggctatgactggattgcagggtctctggacaccagctcttccatcaccagccagcctgaggccttcttctccaagctgcaggagtttcgggaaaccaacaaggaggagtgtatctgcagccatcctgaaccccagttgccaggcctgcgtgagagcagtggcagcggcgtggaggaagaccatgaatgcgtgtactgttaccgtgtcaaccggcgcctgttcccggtgcctgtggatcccggtaccccctgccgcctgtgcaggacaccgcgagaccagcagggccctgggaccctggcgcagccagcgcacgtcagggtgagcatcccgctgtcgatcctggagcccccgcaccggtaccacatccaccggcgaaagagctttgacgcctctgacacactggccctgccccggcactgcctgctgggctgggacatttttcctccgaagtctgagaaaagctcagcccccaggaacctggacctctggtcctctgtctccgctgaggcccagcaccagaagctgtccggcaccagcagcccttttcacccggcctcaccaatgcagatgctgcccccgaccccgacctggtcagtgccccaggtccctcggccccacgtcccacggcagaagccctgaggactgactcctgggggagaacagcattcccgccgcctccagcctctcccctctggcaggcgcacccaggagatggaatcccctgcccgcccagctcaggcccagctgtcctaggttgggcaggtgggtggacccaagcttgtctgctgcctgagttccagagagggaggaccctggggtggagggtgagggattctgtggaagtttgtaaataaagctcagtgctctgcagctc

SEQ ID NO: 8 - protein, 388 aa, Homo sapiens; identical to SEQ ID NO: 1